SEQUENCE LISTING (71 sequences)

SEQ ID NO: 1 - protein, 180 aa, Homo sapiens
  human IL-17B (Propeptide)
MDWPHNLLFLLTISIFLGLGQPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 2 - protein, 160 aa, Homo sapiens
  Human IL-17B (Peptide)
QPRSPKSKRKGQGRPGPLAPGPHQVPLDLVSRMKPYARMEEYERNIEEMVAQLRNSSELAQRKCEVNLQLWMSNKRSLSPWGYSINHDPSRIPVDLPEARCLCLGCVNPFTMQEDRSMVSVPVFSQVPVRRRLCPPPPRTGPCRQRAVMETIAVGCTCIF

SEQ ID NO: 3 - protein, 9 aa, Artificial Sequence
  Epitope 44-52
QVPLDLVSR

SEQ ID NO: 4 - protein, 8 aa, Artificial Sequence
  Epitope 145-152
SQVPVRRR

SEQ ID NO: 5 - protein, 11 aa, Artificial Sequence
  Epitope 155-165
PPPPRTGPCRQ

SEQ ID NO: 6 - protein, 10 aa, Artificial Sequence
  Epitope 84-93
CEVNLQLWMS

SEQ ID NO: 7 - protein, 9 aa, Artificial Sequence
  Epitope 54-62
KPYARMEEY

SEQ ID NO: 8 - protein, 138 aa, Artificial Sequence
  12F9 VH
MGWSWIFLFLLSGTAGVHCQVQLQQSGPELVKPGASVKISCKASGYTFTDYFINWVRQRPGQGLEWIGWIFPGSGSTYYHEKFKGKATLTVDKSSSTAYMLLSSLTSEDSAVYFCARTLYGNWYFDVWGTGTTVTVSS

SEQ ID NO: 9 - protein, 131 aa, Artificial Sequence
  12F9 VL
MKLPVRLLVLMFWIPASSSDVLMTQTPLSLPVSLGDQASISCRSSQSIVHSNGNTYLEWYLQKPGQSPKLLIYKVSNRFSGVPDRFSGSGSGTDFTLKISRVEAEDLGVYYCFQGSHVPYTFGGGTKLEIK

SEQ ID NO: 10 - DNA, 414 bp, Artificial Sequence
  12F9 VH
atgggatggagctggatctttctcttcctcctgtcaggaactgcaggtgtccattgccaggtccagctacagcagtctggacctgagctggtgaagcctggggcttcagtgaagatatcctgcaaggcttctggctacaccttcactgactactttataaactgggtgaggcagaggcctggacagggacttgagtggattggatggatttttcctggaagtggtagtacttactaccatgagaagttcaagggcaaggccacacttactgtagacaaatcctccagcacagcctacatgttgctcagcagcctgacctctgaggactctgcggtctatttctgtgcaagaacgctctatggtaactggtacttcgatgtctggggcacagggaccacggtcaccgtctcctca

SEQ ID NO: 11 - DNA, 393 bp, Artificial Sequence
  12F9 VL
atgaagttgcctgttaggctgttggtgctgatgttctggattcctgcttccagcagtgatgttttgatgacccaaactccactctccctgcctgtcagtcttggagatcaagcctccatctcttgcagatctagtcagagcattgtacatagtaatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtacacgttcggaggggggaccaagctggaaataaaa

SEQ ID NO: 12 - protein, 5 aa, Artificial Sequence
  12F9 VH CDR1
DYFIN

SEQ ID NO: 13 - protein, 17 aa, Artificial Sequence
  12F9 VH CDR2
WIFPGSGSTYYHEKFKG

SEQ ID NO: 14 - protein, 10 aa, Artificial Sequence
  12F9 VH CDR3
TLYGNWYFDV

SEQ ID NO: 15 - protein, 16 aa, Artificial Sequence
  12F9 VL CDR1
RSSQSIVHSNGNTYLE

SEQ ID NO: 16 - protein, 7 aa, Artificial Sequence
  12F9 VL CDR2
KVSNRFS

SEQ ID NO: 17 - protein, 9 aa, Artificial Sequence
  12F9 VL CDR3
FQGSHVPYT

SEQ ID NO: 18 - DNA, 15 bp, Artificial Sequence
  12F9 VH CDR1
gactactttataaac

SEQ ID NO: 19 - DNA, 51 bp, Artificial Sequence
  12F9 VH CDR2
tggatttttcctggaagtggtagtacttactaccatgagaagttcaagggc

SEQ ID NO: 20 - DNA, 30 bp, Artificial Sequence
  12F9 VH CDR3
acgctctatggtaactggtacttcgatgtc

SEQ ID NO: 21 - DNA, 48 bp, Artificial Sequence
  12F9 VL CDR1
agatctagtcagagcattgtacatagtaatggaaacacctatttagaa

SEQ ID NO: 22 - DNA, 21 bp, Artificial Sequence
  12F9 VL CDR2
aaagtttccaaccgattttct

SEQ ID NO: 23 - DNA, 27 bp, Artificial Sequence
  12F9 VL CDR3
tttcaaggttcacatgttccgtacacg

SEQ ID NO: 24 - protein, 137 aa, Artificial Sequence
  13H5 VH
MGRLTSSFLLLIVPAYVLSQVTLKESGPGILQPSQTLSLTCSFSGFSLSTFGMGVGWIRQPSGKGLEWLAHIWWDDDKYYNPALKSRLTISKDTSKNQVFLKIANVDTADTATYYCARMNDGYLYYWGQGTSVTVSS

SEQ ID NO: 25 - protein, 128 aa, Artificial Sequence
  13H5 VL
MDFQVQIFSFLLISASVILSRGQIVLTQSPAIMSASPGEKVTMTCSASSSVSYMYWYQQKPGSSPRLLIYDTSNLASGVPVRFSGSGSGTSYSLTISRMEAEDAATYYCQQWSSYPFTFGSGTKLEIK

SEQ ID NO: 26 - DNA, 411 bp, Artificial Sequence
  13H5 VH
atgggcaggcttacttcttcattcctgttactgattgtccctgcatatgtcctgtcccaggttactctgaaagagtctggccctgggatattgcagccctcccagaccctcagtctgacttgttctttctctgggttttcactgagcacttttggtatgggtgtaggctggattcgtcagccttcagggaagggtctggagtggctggcacacatttggtgggatgatgataagtactataacccagccctgaagagtcggctcacaatctccaaggatacctccaaaaaccaggtattcctcaagatcgccaatgtggacactgcagatactgccacatactactgtgctcgaatgaatgatggttacctatactactggggtcaaggaacctcagtcaccgtctcctca

SEQ ID NO: 27 - DNA, 384 bp, Artificial Sequence
  13H5 VL
atggattttcaagtgcagattttcagcttcctgctaatcagtgcctcagtcatactgtccagaggacaaattgttctcacccagtctccagcaatcatgtctgcatctccaggggagaaggtcaccatgacctgcagtgccagctcaagtgtaagttacatgtactggtaccagcagaagccaggatcctcccccagactcctgatttatgacacatccaacctggcttctggagtccctgttcgcttcagtggcagtgggtctgggacctcttactctctcacaatcagccgaatggaggctgaagatgctgccacttattactgccagcagtggagtagttacccattcacgttcggctcggggacaaagttggaaataaaa

SEQ ID NO: 28 - protein, 7 aa, Artificial Sequence
  13H5 VH CDR1
TFGMGVG

SEQ ID NO: 29 - protein, 16 aa, Artificial Sequence
  13H5 VH CDR2
HIWWDDDKYYNPALKS

SEQ ID NO: 30 - protein, 8 aa, Artificial Sequence
  13H5 VH CDR3
MNDGYLYY

SEQ ID NO: 31 - protein, 10 aa, Artificial Sequence
  13H5 VL CDR1
SASSSVSYMY

SEQ ID NO: 32 - protein, 7 aa, Artificial Sequence
  13H5 VL CDR2
DTSNLAS

SEQ ID NO: 33 - protein, 9 aa, Artificial Sequence
  13H5 VL CDR3
QQWSSYPFT

SEQ ID NO: 34 - DNA, 21 bp, Artificial Sequence
  13H5 VH CDR1
acttttggtatgggtgtaggc

SEQ ID NO: 35 - DNA, 48 bp, Artificial Sequence
  13H5 VH CDR2
cacatttggtgggatgatgataagtactataacccagccctgaagagt

SEQ ID NO: 36 - DNA, 24 bp, Artificial Sequence
  13H5 VH CDR3
atgaatgatggttacctatactac

SEQ ID NO: 37 - DNA, 30 bp, Artificial Sequence
  13H5 VL CDR1
agtgccagctcaagtgtaagttacatgtac

SEQ ID NO: 38 - DNA, 21 bp, Artificial Sequence
  13H5 VL CDR2
gacacatccaacctggcttct

SEQ ID NO: 39 - DNA, 27 bp, Artificial Sequence
  13H5 VL CDR3
cagcagtggagtagttacccattcacg

SEQ ID NO: 40 - protein, 136 aa, Artificial Sequence
  21H6 VH
MGRLTSSFLLLIVPAYVLSQVTLKESGPGILQPSQTLSLTCSFSGFSLSTFGMGVGWIRQPSGKGLEWLAHIWWDDDKYYNPALKGRLTISKDTSKNQVFLKIANVDTADTATYFCARIEDALDYWGQGTSVTVSS

SEQ ID NO: 41 - protein, 127 aa, Artificial Sequence
  21H6 VL
MVSTSQLLGLLLFWTSASRCDIVMTQSPATLSVTPGDRVSLSCRASQNISDYLHWYQQKSHESPRLLIKYTSQSISGIPSRFSGSGSGSDFTLSINSVEPEDVGVYYCQNGHSFPFTFGSGTKLEIK

SEQ ID NO: 42 - DNA, 408 bp, Artificial Sequence
  21H6 VH
atgggcaggcttacttcttcattcctgttactgattgtccctgcatatgtcctgtcccaggttactctgaaagagtctggccctgggatattgcagccctcccagaccctcagtctgacttgttctttctctgggttttcactgagcacttttggtatgggtgtaggctggattcgtcagccttcagggaagggtctggagtggctggcacacatttggtgggatgatgataagtactataacccagccctgaagggtcggctcacaatctccaaggatacctccaaaaaccaggtattcctcaagatcgccaatgtggacactgcagatactgccacatacttctgtgctcgaatagaagatgctttggactactggggtcaaggaacctcagtcaccgtctcctca

SEQ ID NO: 43 - DNA, 381 bp, Artificial Sequence
  21H6 VL
atggtgtccacttctcagctccttggacttttgcttttctggacttcagcctccagatgtgacattgtgatgactcagtctccagccaccctgtctgtgactccaggagatagagtctctctttcctgcagggccagccagaatattagcgactacttacactggtatcagcaaaaatcacatgagtctccaaggcttctcatcaaatatacttcccaatccatctctgggatcccctccaggttcagtggcagtggatcagggtcagatttcactctcagtatcaacagtgtggaacctgaagatgttggagtgtattactgtcaaaatggtcacagctttccattcacgttcggctcggggacaaagttggaaataaaa

SEQ ID NO: 44 - protein, 7 aa, Artificial Sequence
  21H6 VH CDR1
TFGMGVG

SEQ ID NO: 45 - protein, 16 aa, Artificial Sequence
  21H6 VH CDR2
HIWWDDDKYYNPALKG

SEQ ID NO: 46 - protein, 7 aa, Artificial Sequence
  21H6 VH CDR3
IEDALDY

SEQ ID NO: 47 - protein, 11 aa, Artificial Sequence
  21H6 VL CDR1
RASQNISDYLH

SEQ ID NO: 48 - protein, 7 aa, Artificial Sequence
  21H6 VL CDR2
YTSQSIS

SEQ ID NO: 49 - protein, 9 aa, Artificial Sequence
  21H6 VL CDR3
QNGHSFPFT

SEQ ID NO: 50 - DNA, 21 bp, Artificial Sequence
  21H6 VH CDR1
acttttggtatgggtgtaggc

SEQ ID NO: 51 - DNA, 48 bp, Artificial Sequence
  21H6 VH CDR2
cacatttggtgggatgatgataagtactataacccagccctgaagggt

SEQ ID NO: 52 - DNA, 21 bp, Artificial Sequence
  21H6 VH CDR3
atagaagatgctttggactac

SEQ ID NO: 53 - DNA, 33 bp, Artificial Sequence
  21H6 VL CDR1
agggccagccagaatattagcgactacttacac

SEQ ID NO: 54 - DNA, 21 bp, Artificial Sequence
  21H6 VL CDR2
tatacttcccaatccatctct

SEQ ID NO: 55 - DNA, 27 bp, Artificial Sequence
  21H6 VL CDR3
caaaatggtcacagctttccattcacg

SEQ ID NO: 56 - protein, 118 aa, Artificial Sequence
  18G9 VH
QITLKESGPGILKPSQTLSLTCSFSGFSLSTSGMGVGWIRQPSGKGLEWLAHIWWDDDKYYNPSLKSQLTISKDTSRNQVFLRITSVDTADTATYYCARRTQGYFDYWGQGTTLTVSS

SEQ ID NO: 57 - protein, 111 aa, Artificial Sequence
  18G9 VL
DILMTQSPASLAVSLGQRATISCKASQSVDYDGDSYMNWYQQKPGQPPKLLIYAASNLESGIPARFSGSGSGTDFTLNIHPVEEEDAATYYCQQSNEDPLTFGAGTKLELK

SEQ ID NO: 58 - DNA, 354 bp, Artificial Sequence
  18G9 VH
caaattactctaaaagagtctggccctgggatattgaagccctcacagaccctcagtctgacttgttctttctctgggttttcactgagcacttctggtatgggtgtaggctggattcgtcagccttcagggaagggtctggaatggctggcacacatttggtgggatgatgataagtactataacccatccctgaagagccagctcacaatctccaaggatacctccagaaaccaggtattcctcaggatcaccagtgtggacactgcagatactgccacttactactgtgctcgaagaactcaggggtactttgactactggggccaaggcaccactctcacagtctcctca

SEQ ID NO: 59 - DNA, 333 bp, Artificial Sequence
  18G9 VL
gacatcctgatgacccaatctccagcttctttggctgtgtctctagggcagagggccaccatctcctgcaaggccagccaaagtgttgattatgatggtgatagttatatgaactggtaccaacagaaaccaggacagccacccaaactcctcatctatgctgcatccaatctagaatctgggatcccagccaggtttagtggcagtgggtctgggacagacttcaccctcaacatccatcctgtggaggaggaggatgctgcaacctattactgtcagcaaagtaatgaggatcctctcacgttcggtgctgggaccaagctggagctgaaa

SEQ ID NO: 60 - protein, 7 aa, Artificial Sequence
  18G9 VH CDR1
TSGMGVG

SEQ ID NO: 61 - protein, 16 aa, Artificial Sequence
  18G9 VH CDR2
HIWWDDDKYYNPSLKS

SEQ ID NO: 62 - protein, 8 aa, Artificial Sequence
  18G9 VH CDR3
RTQGYFDY

SEQ ID NO: 63 - protein, 15 aa, Artificial Sequence
  18G9 VL CDR1
KASQSVDYDGDSYMN

SEQ ID NO: 64 - protein, 7 aa, Artificial Sequence
  18G9 VL CDR2
AASNLES

SEQ ID NO: 65 - protein, 9 aa, Artificial Sequence
  18G9 VL CDR3
QQSNEDPLT

SEQ ID NO: 66 - DNA, 21 bp, Artificial Sequence
  18G9 VH CDR1
acttctggtatgggtgtaggc

SEQ ID NO: 67 - DNA, 48 bp, Artificial Sequence
  18G9 VH CDR2
cacatttggtgggatgatgataagtactataacccatccctgaagagc

SEQ ID NO: 68 - DNA, 24 bp, Artificial Sequence
  18G9 VH CDR3
agaactcaggggtactttgactac

SEQ ID NO: 69 - DNA, 45 bp, Artificial Sequence
  18G9 VL CDR1
aaggccagccaaagtgttgattatgatggtgatagttatatgaac

SEQ ID NO: 70 - DNA, 21 bp, Artificial Sequence
  18G9 VL CDR2
gctgcatccaatctagaatct

SEQ ID NO: 71 - DNA, 27 bp, Artificial Sequence
  18G9 VL CDR3
cagcaaagtaatgaggatcctctcacg

(19) United States
(12) Patent Application Publication
(10) Pub. No.: US 2022/0281967 A1
(43) Pub. Date: Sep. 8, 2022

(54) NOVEL IL-17B ANTIBODIES

(71) Applicant: OREGA BIOTECH, ECULLY (FR)

(72) Inventors: Jérémy BASTID, TASSIN LA DEMI LUNE (FR); Nathalie BONNEFOY, SAINT CLEMENT DE RIVIERE (FR); Armand BENSUSSAN, PARIS (FR); Gilles ALBERICI, CHARBONNIERES LES BAINS (FR)

(21) Appl. No.: 17/632,370

(22) PCT Filed: Jul. 30, 2020
(86) PCT No.: PCT/EP2020/071547

§ 371 Date: Feb. 2, 2022

(30) Foreign Application Priority Data
Aug. 2, 2019 (EP) 19306004.3

(51) Int. Cl. C07K 16/24; A61K 39/395; C12N 15/64; G01N 33/68; C12P 21/00

(52) U.S. Cl. CPC C07K 16/244; A61K 39/3955; C12N 15/64; G01N 33/6869; C12P 21/005; G01N 2333/54

(57) ABSTRACT

The invention relates to novel IL-17B antagonist antibodies and their use in the diagnosis or treatment of IL-17B mediated diseases.

### BACKGROUND OF THE INVENTION

### Field of the Invention

The invention relates generally to novel IL-17B antagonist antibodies and their use in the diagnosis or treatment of IL-17B mediated diseases.

### Background

The interleukin 17 (IL-17) family comprises 6 interleukins (IL-17A, IL-17B, IL-17C, IL-17D, IL-17E=IL-25 and IL-17F) and their receptors (IL-17RA, IL-17RB, IL-17RC, IL-17RD and IL-17RE) (Gaffen, S. L., 2009). IL-17A exists as a homodimer (IL-17A/A) or heterodimer (IL-17A/F) (Gaffen, S. L., 2009).

IL-17A and IL-17F bind a trimeric complex of IL-17RA and IL-17RC whose expressions are ubiquitous. IL-17A and IL-17F are mainly produced by Th17 cells, a subset of T lymphocytes. The biological role of IL-17A and IL-17F is to participate to host defense against microbial or fungal infections by inducing an acute inflammatory response leading to release of pro-inflammatory cytokines, chemokines, antimicrobial peptides and matrix metalloproteinases from fibroblasts, keratinocytes, endothelial and epithelial cells (Iwakura, Y. et al., 2011). IL-17A also recruits neutrophils to the inflammatory sites (Iwakura, Y. et al., 2011). However, chronic or ectopic production of IL-17A and IL-17F is involved in the development of autoimmune and chronic inflammatory diseases as evidenced in mouse models of Experimental Autoimmune Encephalomyelitis, Collagen-Induced Arthritis (CIA) or SKG arthritic mice, in various models of colitis and psoriasis (Iwakura, Y. et al., 2011). In humans, IL-17A and/or IL-17F are for instance involved in Rheumatoid Arthritis (RA), Multiple Sclerosis, Systemic Lupus Erythematosus inflammatory bowel diseases, Crohn's diseases and psoriasis (Iwakura, Y. et al., 2011). IL-17A triggers a positive feedback loop on IL-6 signaling, including activation of the NFκB and Stat3 in fibroblasts, leading to chronic inflammation. IL-17A and IL-17F are also involved in allergic diseases (Iwakura, Y. et al., 2011).

The 4 other members of the family were identified recently based on sequence homology, but have been poorly studied so far. IL-17B binds IL-17RB; IL-17C binds IL-17RE, IL-17E binds a complex of IL-17RA and IL-17RB; the receptor of IL-17D is unknown (Gaffen, S. L., 2009). IL-17B, IL-17C, IL-17D and IL-17E activate pathways and cytokines release similar to those induced by IL-17A and IL-17F such as NFκb, TNFα, IL-6, IL-8, IL-1β (Lee, J. et al. (2001); Starnes, T. et al. (2002); Stamp, L. K. et al. (2008); Iwakura, Y. et al. (2011); Ramirez-Carrozzi, V. et al. (2011)). As IL-17A and IL-17F, IL-17C is important for the defense against bacterial infections (Ramirez-Carrozzi, V. et al. (2011)). As IL-17A and IL-17F, IL-17B and IL-17C also play a role in CIA in mice (Yamaguchi (2007)) and expression of IL-17A, IL-17B, IL-17D, IL-17E have been detected in human RA nodules (Stamp, L. K. et al. (2008)). IL-17E, originally named IL-25, is the most divergent member of the family. It has some common (NFκB, IL-6, IL-8) and unique (IL-4, IL-5, and IL-13) targets (Wong, C. K. et al. (2005); Iwakura, Y. et al. (2011)). IL-17E is involved in asthma by inducing Th2 cell cytokines (Iwakura, Y. et al. (2011)).

Document WO2013/186236 has confirmed the involvement of the IL-17B pathway in cancers. An increased expression has been observed but also its impact on the effectiveness of chemotherapeutics as well as on cancer cell proliferation and invasion.

Accordingly, antibodies against IL-17B are useful tools for the diagnosis and/or treatment of a large panel of diseases. Novel antibodies against IL-17B with specific and advantageous properties are therefore required, especially those directed against the IL-17B cytokine and having an antagonistic activity.

### DETAILED DESCRIPTION OF THE INVENTION

### Definitions

Unless otherwise expressly defined, the terms used herein are to be understood according to their ordinary meaning in the art. Terms used in the singular or referred to as “a” or “an” also include the plural and vice versa, unless otherwise specified or indicated by context. Standard techniques and procedures are generally performed according to conventional methods in the art and various general references (see generally, Sambrook et al. Molecular Cloning: A Laboratory Manual, 2nd ed. (1989) Cold Spring Harbor Laboratory Press, Cold Spring Harbor, N.Y.), which are provided throughout this document.

As used herein, the term “IL-17B” denotes the IL-17B protein or interleukin 17B, advantageously of human origin. The sequence of the human IL-17B (propeptide) is as set forth in SEQ ID NO: 1. The sequence of the human IL-17B deprived of the signal sequence is as set forth in SEQ ID NO: 2. In the rest of the application, the residues are numbered with reference to SEQ ID NO: 1.

The term “IL-17B antibody” refers to an antibody directed against human IL-17B.

According to the present invention, “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies and antibody fragments. In natural antibodies, two heavy chains are linked to each other by disulfide bonds and each heavy chain is linked to a light chain by a disulfide bond. There are two types of light chain, lambda (l) and kappa (k). There are five main heavy chain classes (or isotypes) which determine the functional activity of an antibody molecule: IgM, IgD, IgG, IgA and IgE. Each chain contains distinct sequence domains. The light chain includes two domains, a variable domain (VL) and a constant domain (CL). The heavy chain includes four domains, a variable domain (VH) and three constant domains (CHI, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR). The Fv fragment is the N-terminal part of the Fab fragment of an immunoglobulin and consists of the variable portions of one light chain and one heavy chain. The specificity of the antibody resides in the structural complementarity between the antibody combining site and the antigenic determinant. Antibody combining sites are made up of residues that are primarily from the hyper variable or complementarity determining regions (CDRs). Occasionally, residues from non hyper variable or framework regions (FR) influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, includes six CDRs, comprising the CDR set from each of a heavy and a light chain V region. Framework Regions (FRs) refer to amino acid sequences interposed between CDRs. This particular region has been described by Kabat et al. (1983) U.S. Dept. of Health and Human Services, “Sequences of Proteins of Immunological Interest” and by Chothia and Lesk, J. Mol. Biol. 196:901-917 (1987), which are incorporated herein by reference, where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues that encompass the CDRs as defined by each of the above cited references are set forth below in Table 1 as a comparison. The exact residue numbers that encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| | Kabat | Chothia |
|---|---|---|
| VH CDR1 | 31-35 | 26-32 |
| VH CDR2 | 50-65 | 52-58 |
| VH CDR3 | 95-102 | 95-102 |
| VL CDR1 | 24-34 | 26-32 |
| VL CDR2 | 50-56 | 50-52 |
| VL CDR3 | 89-97 | 91-96 |

¹Numbering of all CDR definitions in Table 1 is according to the numbering conventions set forth by Kabat et al. (see below).

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al. (1983) U.S. Dept. of Health and Human Services, “Sequence of Proteins of Immunological Interest.” Unless otherwise specified, references to the numbering of specific amino acid residue positions in an IL-17B antibody or antigen-binding fragment, variant, or derivative thereof of the present invention are according to the Kabat numbering system. It is noted that sequences presented in the accompanying SEQUENCE LISTING are not numbered according to Kabat, but it is well within the ordinary skill in the art to determine the Kabat numbering of sequences in the SEQUENCE LISTING.

As used herein, the term “antigen-binding fragment” refers to one or more fragments of an antibody that retain the ability to specifically bind to an antigen (e.g., IL-17B). The antigen-binding function of an antibody can be performed by fragments of a full antibody. Examples of “antigen-binding fragments” include (i) an Fab fragment, a monovalent fragment consisting of the V_{L}, V_{H}, C_{L} and C_{H}1 domains; (ii) an F(ab)₂ fragment, a bivalent fragment comprising two Fab fragments linked by a disulfide bridge at the hinge region; (iii) an Fab′ fragment, an Fab with part of the hinge region (see, FUNDAMENTAL IMMUNOLOGY, Paul ed., 3r^{d} ed. 1993); (iv) an Fd fragment, consisting of the V_{H} and C_{H}1 domains; (v) an Fv fragment consisting of the V_{L} and V_{H} domains of a single arm of an antibody, (vi) a dAb fragment (Ward et al., (1989) Nature 341:544-546), which consists of a V_{H} domain; (vii) an isolated complementarity determining region (CDR); and (viii) a nanobody, a heavy chain variable region containing a single variable domain and two constant domains. Furthermore, although the two domains of the Fv fragment, V_{L} and V_{H}, are encoded by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

As used herein, the term “monoclonal antibody” refers to a preparation of antibody molecules of single molecular composition. A monoclonal antibody composition displays a single binding specificity and affinity for a particular epitope of an antigen (i.e., IL-17B).

As used herein, the term “human antibody” is intended to include antibodies having variable regions in which both the framework and CDR regions are derived from human germline immunoglobulin sequences. If the antibody contains a constant region, the constant region also is derived from human germline immunoglobulin sequences. Human antibodies for use in the invention may include amino acid residues not encoded by human germline immunoglobulin sequences (e.g., mutations introduced by random or site-specific mutagenesis in vitro or by somatic mutation in vivo).

The term “chimeric antibody” refers to an antibody which comprises a VH domain and a VL domain of an antibody derived from the isolated murine antibody, and a CH domain and a CL domain of a human antibody.

According to the invention, the term “humanized antibody” refers to an antibody having variable region framework and constant regions from a human antibody (e. g., an “acceptor” antibody) but retains the CDRs and optionally, select framework residues of the isolated murine antibody (e.g., the “parent” antibody).

The term “Fab” denotes an antibody fragment having a molecular weight of about 50,000 and antigen binding activity, in which about a half of the N-terminal side of H chain and the entire L chain, among fragments obtained by treating IgG with a protease, papaine, are bound together through a disulfide bond.

The term “F(ab′)2” refers to an antibody fragment having a molecular weight of about 100,000 and antigen binding activity, which is slightly larger than the Fab bound via a disulfide bond of the hinge region, among fragments obtained by treating IgG with a protease, pepsin.

The term “Fab′” refers to an antibody fragment having a molecular weight of about 50,000 and antigen binding activity, which is obtained by cutting a disulfide bond of the hinge region of the F(ab′)2.

A single chain Fv (“scFv”) polypeptide is a covalently linked VH::VL heterodimer which is usually expressed from a gene fusion including VH and VL encoding genes linked by a peptide-encoding linker. “dsFv” is a VH::VL heterodimer stabilised by a disulfide bond. Divalent and multivalent antibody fragments can form either spontaneously by association of monovalent scFvs, or can be generated by coupling monovalent scFvs by a peptide linker, such as divalent sc(Fv)2.

The term “diabodies” refers to small antibody fragments with two antigen binding sites, which fragments comprise a heavy-chain variable domain (VH) connected to a light-chain variable domain (VL) in the same polypeptide chain (VH-VL). By using a linker that is too short to allow pairing between the two domains on the same chain, the domains are forced to pair with the complementary domains of another chain and create two antigen-binding sites.

By “purified” and “isolated” it is meant, when referring to a polypeptide (i.e. an antibody according to the invention) or to a nucleotide sequence, that the indicated molecule is present in the substantial absence of other biological macromolecules of the same type. The term “purified” as used herein preferably means at least 75% by weight, more preferably at least 85% by weight, more preferably still at least 95% by weight, and most preferably at least 98% by weight, of biological macromolecules of the same type are present. An “isolated” nucleic acid molecule which encodes a particular polypeptide refers to a nucleic acid molecule which is substantially free of other nucleic acid molecules that do not encode the polypeptide; however, the molecule may include some additional bases or moieties which do not deleteriously affect the basic characteristics of the composition.

As used herein, the term “subject” or “patient” refers to a mammalian animal (including but not limited to non-primates such as cows, pigs, horses, sheep, cows, dogs, cats, rats, and mice), more specifically a primate (including but not limited to monkeys, apes, and humans), and even more specifically, a human.

In the context of the invention, the term “treating” or “treatment”, as used herein, means reversing, alleviating, inhibiting the progress of, or preventing the disorder or condition to which such term applies, or one or more symptoms of such disorder or condition. A “therapeutically effective amount” is intended for a minimal amount of active agent which is necessary to impart therapeutic benefit to a subject. For example, a “therapeutically effective amount” to a mammal is such an amount which induces, ameliorates or otherwise causes an improvement in the pathological symptoms, disease progression or physiological conditions associated with or resistance to succumbing to a disorder. The amount of a therapeutic agent that is effective to alleviate any particular disease symptom or effect (also referred to as the “therapeutically effective amount”) or prevent a particular disease symptom or effect (also referred to as the “prophylactically effective amount”) may vary according to factors such as the disease state, age, and weight of the patient, and the ability of the drug to elicit a desired response in the patient. Whether a disease symptom or effect has been alleviated can be assessed by any clinical measurement typically used (e.g., by healthcare providers or laboratory clinicians) to assess the severity or progression status of that symptom or effect.

As used herein, the term “sample” refers any biological fluid, cell, tissue, or other component from a subject. Samples include, without limitation, tissue fragments (diseased or non-diseased), organs, cells, cellular components, whole blood, serum, plasma, saliva, urine, synovial fluid, bone marrow, cerebrospinal fluid, vaginal mucus, cervical mucus, nasal secretions, sputum, semen, amniotic fluid, bronchoalveolar lavage fluid, cellular exudates, and tumor fragments.

### Description of the Antibodies of the Invention

The present invention relates to novel isolated IL-17B antibodies. The invention also relates to antibodies which bind the same epitope(s) or which comprise a variable light chain (VL) comprising the CDRs of the VL chain of said antibodies and a variable heavy chain (VH) comprising the CDRs of the VH chain of said antibodies, respectively. The invention also relates to the use of said antibodies in the diagnosis, prevention or treatment of IL-17B mediated diseases and disorders.

The inventors have identified a crucial epitope on the human IL-17B cytokine of relevance for antibody binding as well as neutralizing activity, but also further secondary epitopes increasing the performances of the antibodies able to bind to the main epitope.

According to one aspect, the present invention relates to an isolated IL-17B antibody which binds a sequence of the human IL-17B as set forth in SEQ ID NO: 3. In other words, the present invention relates to an isolated IL-17B antibody which binds as a major epitope a sequence comprising or consisting of SEQ ID NO: 3. According to a preferred embodiment, an antibody according to the invention further binds at least another sequence selected in the group consisting of SEQ ID NO: 4, SEQ ID NO: 5 and SEQ ID NO: 6, advantageously one of sequence SEQ ID NO: 4 and one of sequence SEQ ID NO: 5. In other words, the present invention relates to an isolated IL-17B antibody which binds as a further epitope at least a sequence comprising or consisting of SEQ ID NO: 4, SEQ ID NO: 5 and/or SEQ ID NO: 6, advantageously SEQ ID NO: 4 and SEQ ID NO: 5.

The present invention further provides examples of isolated anti-IL-17B antibodies or antigen-binding fragments, variants, or derivatives thereof that bind to IL-17B, e.g., murine monoclonal antibodies (mAbs) 12F9, 13H5, 21H6 and 18G9. In certain embodiments, the anti-IL-17B antibodies bind IL-17B, especially human IL-17B, advantageously of sequence SEQ ID NO: 1 or SEQ ID NO: 2. In some embodiments, the anti-IL-17B antibodies have a neutralizing effect on the activity of IL-17B. In one embodiment, neutralizing activity of the antibody is measured by ability to block secretion of IL-8 by HepG2 cells although other assays known to those of skill in the art could also be used. In other words, the antibody of the invention is an antagonist of the IL-17B cytokine, advantageously an antagonist of an human IL-17B polypeptide. According to a preferred embodiment, the present invention relates to an isolated antagonist antibody, wherein said antibody is monoclonal antibody 12F9, 13H5, 21H6 or 18G9, advantageously 13H5 or 21H6.

According to another aspect, the present invention relates to an isolated antibody that specifically binds to the same IL-17B epitope as reference monoclonal antibody 12F9, 13H5, 21H6 or 18G9. According to another aspect, the present invention relates to an isolated antibody that specifically binds to IL-17B, wherein said antibody competitively inhibits reference monoclonal antibody 12F9, 13H5, 21H6 or 18G9 from specifically binding to IL-17B.

In another embodiment, the antibody of the invention comprises a variable light chain (VL) comprising the CDRs of the VL chain of the 12F9, 13H5, 21H6 or 18G9 antibody, and/or a variable heavy chain (VH) comprising the CDRs of the VH chain of the 12F9, 13H5, 21H6 or 18G9 antibody.

In another embodiment, the antibody of the invention comprises the VL chain of the 12F9, 13H5, 21H6 or 18G9 antibody, and/or the VH chain of the 12F9, 13H5, 21H6 or 18G9 antibody.

The inventors have cloned and characterized the variable domain of the light and heavy chains of these 4 antibodies (12F9, 13H5, 21H6 and 18G9), which all act as neutralizing antibody or antagonist of IL-17B. The corresponding sequences are shown in Table 2 below.

TABLE 2
Amino acid sequences of the
neutralizing antibodies of the invention

| | SEQ | Chain/ | |
|---|---|---|---|
| mAb | ID NO: | CDR | Amino Acid Sequence |
| 12F9 | 8 | VH | See sequence Listing |
| 12F9 | 12 | VH CDR1 | DYFIN |
| 12F9 | 13 | VH CDR2 | WIFPGSGSTYYHEKFKG |
| 12F9 | 14 | VH CDR3 | TLYGNWYFDV |
| 12F9 | 9 | VL | See sequence Listing |
| 12F9 | 15 | VL CDR1 | RSSQSIVHSNGNTYLE |
| 12F9 | 16 | VL CDR2 | KVSNRFS |
| 12F9 | 17 | VL CDR3 | FQGSHVPYT |
| 13H5 | 24 | VH | See sequence Listing |
| 13H5 | 28 | VH CDR1 | TFGMGVG |
| 13H5 | 29 | VH CDR2 | HIWWDDDKYYNPALKS |
| 13H5 | 30 | VH CDR3 | MNDGYLYY |
| 13H5 | 25 | VL | See sequence Listing |
| 13H5 | 31 | VL CDR1 | SASSSVSYMY |
| 13H5 | 32 | VL CDR2 | DTSNLAS |
| 13H5 | 33 | VL CDR3 | QQWSSYPFT |
| 21H6 | 40 | VH | See sequence Listing |
| 21H6 | 44 | VH CDR1 | TFGMGVG |
| 21H6 | 45 | VH CDR2 | HIWWDDDKYYNPALKG |
| 21H6 | 46 | VH CDR3 | IEDALDY |
| 21H6 | 41 | VL | See sequence Listing |
| 21H6 | 47 | VL CDR1 | RASQNISDYLH |
| 21H6 | 48 | VL CDR2 | YTSQSIS |
| 21H6 | 49 | VL CDR3 | QNGHSFPFT |
| 18G9a | 56 | VH | See sequence Listing |
| 18G9 | 60 | VH CDR1 | TSGMGVG |
| 18G9 | 61 | VH CDR2 | HIWWDDDKYYNPSLKS |
| 18G9 | 62 | VH CDR3 | RTQGYFDY |
| 18G9 | 57 | VL | See sequence Listing |
| 18G9 | 63 | VL CDR1 | KASQSVDYDGDSYMN |
| 18G9 | 64 | VL CDR2 | AASNLES |
| 18G9 | 65 | VL CDR3 | QQSNEDPLT |

According to a first embodiment (a), the present invention relates to an isolated IL-17B antibody which comprises:
a Kabat heavy chain complementarity determining region-1 (VH-CDR1) amino acid sequence which is identical to the VH-CDR1 of the VH region comprising the amino acid sequence SEQ ID NO: 8; and a Kabat heavy chain complementarity determining region-2 (VH-CDR2) amino acid sequence which is identical to the VH-CDR2 of the VH region comprising the amino acid sequence SEQ ID NO: 8; and a Kabat heavy chain complementarity determining region-3 (VH-CDR3) amino acid sequence which is identical to the VH-CDR3 of the VH region comprising the amino acid sequence SEQ ID NO: 8; and a Kabat light chain complementarity determining region-1 (VL-CDR1) amino acid sequence which is identical to the VL-CDR1 of the VL region comprising the amino acid sequence SEQ ID NO: 9; and a Kabat light chain complementarity determining region-2 (VL-CDR2) amino acid sequence which is identical to the VL-CDR2 of the VL region comprising the amino acid sequence SEQ ID NO: 9; and a Kabat light chain complementarity determining region-3 (VL-CDR3) amino acid sequence which is identical to the VL-CDR3 of the VL region comprising the amino acid sequence SEQ ID NO: 9; or
a Kabat heavy chain complementarity determining region-1 (VH-CDR1) comprising the amino acid sequence SEQ ID NO: 12; and a Kabat heavy chain complementarity determining region-2 (VH-CDR2) comprising the amino acid sequence SEQ ID NO: 13; and a Kabat heavy chain complementarity determining region-3 (VH-CDR3) comprising the amino acid sequence SEQ ID NO: 14; and a Kabat light chain complementarity determining region-1 (VL-CDR1) comprising the amino acid sequence SEQ ID NO: 15; and a Kabat light chain complementarity determining region-2 (VL-CDR2) comprising the amino acid sequence SEQ ID NO: 16; and a Kabat light chain complementarity determining region-3 (VL-CDR3) comprising the amino acid sequence SEQ ID NO: 17; or
a VH chain comprising the amino acid sequence SEQ ID NO: 8; and a VL chain comprising the amino acid sequence SEQ ID NO: 9.

According to a second embodiment (b), the present invention relates to an isolated IL-17B antibody which comprises:
a Kabat heavy chain complementarity determining region-1 (VH-CDR1) amino acid sequence which is identical to the VH-CDR1 of the VH region comprising the amino acid sequence SEQ ID NO: 24; and a Kabat heavy chain complementarity determining region-2 (VH-CDR2) amino acid sequence which is identical to the VH-CDR2 of the VH region comprising the amino acid sequence SEQ ID NO:24; and a Kabat heavy chain complementarity determining region-3 (VH-CDR3) amino acid sequence which is identical to the VH-CDR3 of the VH region comprising the amino acid sequence SEQ ID NO: 24; and a Kabat light chain complementarity determining region-1 (VL-CDR1) amino acid sequence which is identical to the VL-CDR1 of the VL region comprising the amino acid sequence SEQ ID NO: 25; and a Kabat light chain complementarity determining region-2 (VL-CDR2) amino acid sequence which is identical to the VL-CDR2 of the VL region comprising the amino acid sequence SEQ ID NO: 25; and a Kabat light chain complementarity determining region-3 (VL-CDR3) amino acid sequence which is identical to the VL-CDR3 of the VL region comprising the amino acid sequence SEQ ID NO: 25; or
a Kabat heavy chain complementarity determining region-1 (VH-CDR1) comprising the amino acid sequence SEQ ID NO: 28; and a Kabat heavy chain complementarity determining region-2 (VH-CDR2) comprising the amino acid sequence SEQ ID NO: 29; and a Kabat heavy chain complementarity determining region-3 (VH-CDR3) comprising the amino acid sequence SEQ ID NO: 30; and a Kabat light chain complementarity determining region-1 (VL-CDR1) comprising the amino acid sequence SEQ ID NO: 31; and a Kabat light chain complementarity determining region-2 (VL-CDR2) comprising the amino acid sequence SEQ ID NO: 32; and a Kabat light chain complementarity determining region-3 (VL-CDR3) comprising the amino acid sequence SEQ ID NO: 33; or
a VH chain comprising the amino acid sequence SEQ ID NO: 24; and a VL chain comprising the amino acid sequence SEQ ID NO: 25.

According to a third embodiment (c), the present invention relates to an isolated IL-17B antibody which comprises:
a Kabat heavy chain complementarity determining region-1 (VH-CDR1) amino acid sequence which is identical to the VH-CDR1 of the VH region comprising the amino acid sequence SEQ ID NO: 40; and a Kabat heavy chain complementarity determining region-2 (VH-CDR2) amino acid sequence which is identical to the VH-CDR2 of the VH region comprising the amino acid sequence SEQ ID NO: 40; and a Kabat heavy chain complementarity determining region-3 (VH-CDR3) amino acid sequence which is identical to the VH-CDR3 of the VH region comprising the amino acid sequence SEQ ID NO: 40; and a Kabat light chain complementarity determining region-1 (VL-CDR1) amino acid sequence which is identical to the VL-CDR1 of the VL region comprising the amino acid sequence SEQ ID NO: 41; and a Kabat light chain complementarity determining region-2 (VL-CDR2) amino acid sequence which is identical to the VL-CDR2 of the VL region comprising the amino acid sequence SEQ ID NO: 41; and a Kabat light chain complementarity determining region-3 (VL-CDR3) amino acid sequence which is identical to the VL-CDR3 of the VL region comprising the amino acid sequence SEQ ID NO: 41; or
a Kabat heavy chain complementarity determining region-1 (VH-CDR1) comprising the amino acid sequence SEQ ID NO: 44; and a Kabat heavy chain complementarity determining region-2 (VH-CDR2) comprising the amino acid sequence SEQ ID NO: 45; and a Kabat heavy chain complementarity determining region-3 (VH-CDR3) comprising the amino acid sequence SEQ ID NO: 46; and a Kabat light chain complementarity determining region-1 (VL-CDR1) comprising the amino acid sequence SEQ ID NO: 47; and a Kabat light chain complementarity determining region-2 (VL-CDR2) comprising the amino acid sequence SEQ ID NO: 48; and a Kabat light chain complementarity determining region-3 (VL-CDR3) comprising the amino acid sequence SEQ ID NO: 49; or
a VH chain comprising the amino acid sequence SEQ ID NO: 40; and a VL chain comprising the amino acid sequence SEQ ID NO: 41.

According to a fourth embodiment (d), the present invention relates to an isolated IL-17B antibody which comprises:
a Kabat heavy chain complementarity determining region-1 (VH-CDR1) amino acid sequence which is identical to the VH-CDR1 of the VH region comprising the amino acid sequence SEQ ID NO: 56; and a Kabat heavy chain complementarity determining region-2 (VH-CDR2) amino acid sequence which is identical to the VH-CDR2 of the VH region comprising the amino acid sequence SEQ ID NO: 56; and a Kabat heavy chain complementarity determining region-3 (VH-CDR3) amino acid sequence which is identical to the VH-CDR3 of the VH region comprising the amino acid sequence SEQ ID NO: 56; and a Kabat light chain complementarity determining region-1 (VL-CDR1) amino acid sequence which is identical to the VL-CDR1 of the VL region comprising the amino acid sequence SEQ ID NO: 57; and a Kabat light chain complementarity determining region-2 (VL-CDR2) amino acid sequence which is identical to the VL-CDR2 of the VL region comprising the amino acid sequence SEQ ID NO: 57; and a Kabat light chain complementarity determining region-3 (VL-CDR3) amino acid sequence which is identical to the VL-CDR3 of the VL region comprising the amino acid sequence SEQ ID NO: 57; or
a Kabat heavy chain complementarity determining region-1 (VH-CDR1) comprising the amino acid sequence SEQ ID NO: 60; and a Kabat heavy chain complementarity determining region-2 (VH-CDR2) comprising the amino acid sequence SEQ ID NO: 61; and a Kabat heavy chain complementarity determining region-3 (VH-CDR3) comprising the amino acid sequence SEQ ID NO: 62; and a Kabat light chain complementarity determining region-1 (VL-CDR1) comprising the amino acid sequence SEQ ID NO: 63; and a Kabat light chain complementarity determining region-2 (VL-CDR2) comprising the amino acid sequence SEQ ID NO: 64; and a Kabat light chain complementarity determining region-3 (VL-CDR3) comprising the amino acid sequence SEQ ID NO: 65; or
a VH chain comprising the amino acid sequence SEQ ID NO: 56; and a VL chain comprising the amino acid sequence SEQ ID NO: 57.

According to a further aspect, the present invention relates to an isolated IL-17B antibody which binds to the same IL-17B epitope(s) as the isolated IL-17B antibody according to embodiment (a), (b), (c) or (d). According to another aspect, the present invention relates to an isolated antibody that specifically binds to IL-17B, wherein said antibody competitively inhibits reference monoclonal antibody according to embodiment (a), (b), (c) or (d).

According to another aspect, the present invention relates to an isolated antibody that specifically binds to IL-17B, wherein the VH of said antibody comprises a Kabat heavy chain complementarity determining region-1 (VH-CDR1) amino acid sequence which is identical, or identical except for conservative amino acid substitutions, or 80%, 85%, 90%, 91%, 92%, 93%, 94%, 95%, 96%, 97%, 98% or 99% identical to a sequence selected in the group consisting of: SEQ ID NO: 12, SEQ ID NO: 28, SEQ ID NO: 44 and SEQ ID NO: 60.

According to another aspect, the present invention relates to an isolated antibody that specifically binds to IL-17B, wherein the VH of said antibody comprises a Kabat heavy chain complementarity determining region-2 (VH-CDR2) amino acid sequence which is identical, or identical except for conservative amino acid substitutions, or 80%, 85%, 90%, 91%, 92%, 93%, 94%, 95%, 96%, 97%, 98% or 99% identical to a sequence selected in the group consisting of: SEQ ID NO: 13, SEQ ID NO: 29, SEQ ID NO: 45 and SEQ ID NO: 61.

According to another aspect, the present invention relates to an isolated antibody that specifically binds to IL-17B, wherein the VH of said antibody comprises a Kabat heavy chain complementarity determining region-3 (VH-CDR3) amino acid sequence which is identical, or identical except for conservative amino acid substitutions, or 80%, 85%, 90%, 91%, 92%, 93%, 94%, 95%, 96%, 97%, 98% or 99% identical to a sequence selected in the group consisting of: SEQ ID NO: 14, SEQ ID NO: 30, SEQ ID NO: 46 and SEQ ID NO: 62.

According to another aspect, the present invention relates to an isolated antibody that specifically binds to IL-17B, wherein the VL of said antibody comprises a Kabat light chain complementarity determining region-1 (VL-CDR1) amino acid sequence which is identical, or identical except for conservative amino acid substitutions, or 80%, 85%, 90%, 91%, 92%, 93%, 94%, 95%, 96%, 97%, 98% or 99% identical to a sequence selected in the group consisting of: SEQ ID NO: 15, SEQ ID NO: 31, SEQ ID NO: 47, and SEQ ID NO: 63.

According to another aspect, the present invention relates to an isolated antibody that specifically binds to IL-17B, wherein the VL of said antibody comprises a Kabat light chain complementarity determining region-2 (VL-CDR2) amino acid sequence which is identical, or identical except for conservative amino acid substitutions, or 80%, 85%, 90%, 91%, 92%, 93%, 94%, 95%, 96%, 97%, 98% or 99% identical to a sequence selected in the group consisting of: SEQ ID NO: 16, SEQ ID NO: 32, SEQ ID NO: 48, and SEQ ID NO: 64.

According to another aspect, the present invention relates to an isolated antibody that specifically binds to IL-17B, wherein the VL of said antibody comprises a Kabat light chain complementarity determining region-3 (VL-CDR3) amino acid sequence which is identical, or identical except for conservative amino acid substitutions, or 80%, 85%, 90%, 91%, 92%, 93%, 94%, 95%, 96%, 97%, 98% or 99% identical to a sequence selected in the group consisting of: SEQ ID NO: 17, SEQ ID NO: 33, SEQ ID NO: 49, and SEQ ID NO: 65.

According to another aspect, the present invention relates to an isolated antibody that specifically binds to IL-17B, wherein the VH of said antibody comprises or consists of an amino acid sequence which is identical, or identical except for conservative amino acid substitutions, or 80%, 85%, 90%, 91%, 92%, 93%, 94%, 95%, 96%, 97%, 98% or 99% identical to a sequence selected in the group consisting of: SEQ ID NO: 8, SEQ ID NO: 24, SEQ ID NO: 40 and SEQ ID NO: 56.

According to another aspect, the present invention relates to an isolated antibody that specifically binds to IL-17B, wherein the VL of said antibody comprises or consists of an amino acid sequence which is identical, or identical except for conservative amino acid substitutions, or 80%, 85%, 90%, 91%, 92%, 93%, 94%, 95%, 96%, 97%, 98% or 99% identical to a sequence selected in the group consisting of: SEQ ID NO: 9, SEQ ID NO: 25, SEQ ID NO: 41 and SEQ ID NO: 57.

Antibodies 12F9, 13H5, 21H6 and 18G9 all recognize, as a major epitope, the sequence QVPLDLVSR as shown in SEQ ID NO: 3. This newly identified sequence corresponds to residues 44 to 52 of the human IL-17B sequence, as set forth in SEQ ID NO: 1. A second epitope, the sequence SQVPVRRR (SEQ ID NO: 4), can be found in the IL-17B polypeptide as set forth in SEQ ID NO: 1 and corresponds to residues 145 to 152. A third epitope, the sequence PPPPRTGPCRQ (SEQ ID NO: 5), can be found in the IL-17B polypeptide as set forth in SEQ ID NO: 1 and corresponds to residues 155 to 165. A fourth epitope, the sequence CEVNLQLWMS (SEQ ID NO: 6), can be found in the IL-17B polypeptide as set forth in SEQ ID NO: 1 and corresponds to residues 84 to 93. The second and third epitopes are recognized by antibodies 13H5, 21H6 and 18G9. The fourth epitope is recognized only by antibody 18G9. In the frame of the invention, the major epitope is defined as the region of the antigen which shows the strongest binding with the tested antibody. These epitopes are the core binding regions. Adjacent regions are often also involved in giving the epitope the correct structure.

Another aspect of the invention concerns any IL-17B antibody having, as the major antibody binding epitope, the same epitope as reference antibodies 12F9, 13H5, 21H6 or 18G9. A further aspect of the invention concerns any IL-17B antibody having, as a major antibody binding epitope, the sequence QVPLDLVSR (SEQ ID NO: 3) of the human IL-17B protein formed by amino acid residues 44 to 52 of sequence SEQ ID NO: 1.

Antibodies of the invention can be produced by any technique well known in the art. In particular said antibodies are produced by techniques as hereinafter described. Polyclonal as well as monoclonal antibodies are concerned by the present invention. Monoclonal antibodies (mAb) are preferred.

In another embodiment, an antibody of the invention is a chimeric antibody, preferably a chimeric mouse/human antibody. In particular, said mouse/human chimeric antibody may comprise the variable domains of the antibodies according to the invention.

In another embodiment, an antibody of the invention is a humanized antibody. In particular, in said humanized antibody, the variable domain comprises human acceptor frameworks regions, and optionally human constant domain where present, and non-human donor CDRs, such as mouse CDRs as defined above. In another embodiment, an antibody of the invention is a human antibody.

The invention further provides fragments of said antibodies which include but are not limited to Fv, Fab, F(ab′)2, Fab′, dsFv, scFv, sc(Fv)2 and diabodies; and multispecific antibodies formed from antibody fragments.

In an embodiment, the antibody of the invention comprises a light chain constant region selected from the group consisting of a human kappa constant region and a human lambda constant region. In another embodiment, the antibody of the invention comprises a heavy chain constant region or fragment thereof, advantageously human IgG1, IgG2, IgG3, IgG4, IgM, IgA1, IgA2, IgE or IgD.

In certain embodiments, the antibodies and polypeptides of the invention can be used in an isolated (e.g., purified) form or contained in a vector, such as a membrane or lipid vesicle (e.g. a liposome).

### Nucleic Acids, Vectors and Recombinant Host Cells of the Invention

A further object of the invention relates to a nucleic acid sequence encoding an antibody of the invention or a fragment thereof as disclosed above.

In a particular embodiment, the invention relates to a nucleic acid sequence encoding the VH domain, VL domain or CDR regions of mAbs 12F9, 13H5, 21H6 and 18G9, as shown in Table 3 below:

TABLE 3
Nucleotide sequences encoding the neutralizing antibodies of the invention

| mAb | SEQ ID NO: | Chain/CDR | Amino Acid Sequence |
|---|---|---|---|
| 12F9 | 10 | VH | See sequence Listing |
| 12F9 | 18 | VH CDR1 | gactacttta taaac |
| 12F9 | 19 | VH CDR2 | tggatttttc ctggaagtgg tagtacttac taccatgaga |
| | | | agttcaaggg c |
| 12F9 | 20 | VH CDR3 | acgctctatg gtaactggta cttcgatgtc |
| 12F9 | 11 | VL | See sequence Listing |
| 12F9 | 21 | VL CDR1 | agatctagtc agagcattgt acatagtaat |
| | | | ggaaacacct atttagaa |
| 12F9 | 22 | VL CDR2 | aaagtttcca accgattttc t |
| 12F9 | 23 | VL CDR3 | tttcaaggtt cacatgttcc gtacacg |
| 13H5 | 26 | VH | See sequence Listing |
| 13H5 | 34 | VH CDR1 | acttttggta tgggtgtagg c |
| 13H5 | 35 | VH CDR2 | cacatttggt gggatgatga taagtactat |
| | | | aacccagccc tgaagagt |
| 13H5 | 36 | VH CDR3 | atgaatgatg gttacctata ctac |
| 13H5 | 27 | VL | See sequence Listing |
| 13H5 | 37 | VL CDR1 | agtgccagct caagtgtaag ttacatgtac |
| 13H5 | 38 | VL CDR2 | gacacatcca acctggcttc t |
| 13H5 | 39 | VL CDR3 | cagcagtgga gtagttaccc attcacg |
| 21H6 | 42 | VH | See sequence Listing |
| 21H6 | 50 | VH CDR1 | acttttggta tgggtgtagg c |
| 21H6 | 51 | VH CDR2 | acatttggt gggatgatga taagtactat aacccagccc |
| | | | tgaagggt |
| 21H6 | 52 | VH CDR3 | atagaagatg ctttggacta c |
| 21H6 | 43 | VL | See sequence Listing |
| 21H6 | 53 | VL CDR1 | agggccagcc agaatattag cgactactta cac |
| 21H6 | 54 | VL CDR2 | tatacttccc aatccatctc t |
| 21H6 | 55 | VL CDR3 | caaaatggtc acagctttcc attcacg |
| 18G9 | 58 | VH | See sequence Listing |
| 18G9 | 66 | VH CDR1 | acttctggta tgggtgtagg c |
| 18G9 | 67 | VH CDR2 | cacatttggt gggatgatga taagtactat |
| | | | aacccatccc tgaagagc |
| 18G9 | 68 | VH CDR3 | agaactcagg ggtactttga ctac |
| 18G9 | 59 | VL | See sequence Listing |
| 18G9 | 69 | VL CDR1 | aaggccagcc aaagtgttga ttatgatggt |
| | | | gatagttata tgaac |
| 18G9 | 70 | VL CDR2 | gctgcatcca atctagaatc t |
| 18G9 | 71 | VL CDR3 | cagcaaagta atgaggatcc tctcacg |

In one aspect, the invention relates to a polynucleotide that is identical or 80%, 85%, 90%, 91%, 92%, 93%, 94%, 95%, 96%, 97%, 98% or 99% identical to a sequence selected in the group consisting of: SEQ ID NO: 10, SEQ ID NO: 11, SEQ ID NO: 18 to SEQ ID NO: 23, SEQ ID NO: 26, SEQ ID NO: 27, SEQ ID NO: 34 to SEQ ID NO: 39, SEQ ID NO: 42, SEQ ID NO: 43, SEQ ID NO: 50 to SEQ ID NO: 55, SEQ ID NO: 58, SEQ ID NO: 59, SEQ ID NO: 66 to SEQ ID NO: 71.

According to a first embodiment (a), the present invention relates to a polynucleotide encoding an isolated IL-17B antibody, wherein said polynucleotide encodes:
a Kabat heavy chain complementarity determining region-1 (VH-CDR1) amino acid sequence which is identical to the VH-CDR1 of the VH region comprising the amino acid sequence SEQ ID NO: 8; and a Kabat heavy chain complementarity determining region-2 (VH-CDR2) amino acid sequence which is identical to the VH-CDR2 of the VH region comprising the amino acid sequence SEQ ID NO: 8; and a Kabat heavy chain complementarity determining region-3 (VH-CDR3) amino acid sequence which is identical to the VH-CDR3 of the VH region comprising the amino acid sequence SEQ ID NO: 8; and a Kabat light chain complementarity determining region-1 (VL-CDR1) amino acid sequence which is identical to the VL-CDR1 of the VL region comprising the amino acid sequence SEQ ID NO: 9; and a Kabat light chain complementarity determining region-2 (VL-CDR2) amino acid sequence which is identical to the VL-CDR2 of the VL region comprising the amino acid sequence SEQ ID NO: 9; and a Kabat light chain complementarity determining region-3 (VL-CDR3) amino acid sequence which is identical to the VL-CDR3 of the VL region comprising the amino acid sequence SEQ ID NO: 9; or
a Kabat heavy chain complementarity determining region-1 (VH-CDR1) comprising the amino acid sequence SEQ ID NO: 12; and a Kabat heavy chain complementarity determining region-2 (VH-CDR2) comprising the amino acid sequence SEQ ID NO: 13; and a Kabat heavy chain complementarity determining region-3 (VH-CDR3) comprising the amino acid sequence SEQ ID NO: 14; and a Kabat light chain complementarity determining region-1 (VL-CDR1) comprising the amino acid sequence SEQ ID NO: 15; and a Kabat light chain complementarity determining region-2 (VL-CDR2) comprising the amino acid sequence SEQ ID NO: 16; and a Kabat light chain complementarity determining region-3 (VL-CDR3) comprising the amino acid sequence SEQ ID NO: 17; or
a VH chain comprising the amino acid sequence SEQ ID NO: 8; and a VL chain comprising the amino acid sequence SEQ ID NO: 9.

According to a second embodiment (b), the present invention relates to a polynucleotide encoding an isolated IL-17B antibody, wherein said polynucleotide encodes:
a Kabat heavy chain complementarity determining region-1 (VH-CDR1) amino acid sequence which is identical to the VH-CDR1 of the VH region comprising the amino acid sequence SEQ ID NO: 24; and a Kabat heavy chain complementarity determining region-2 (VH-CDR2) amino acid sequence which is identical to the VH-CDR2 of the VH region comprising the amino acid sequence SEQ ID NO: 24; and a Kabat heavy chain complementarity determining region-3 (VH-CDR3) amino acid sequence which is identical to the VH-CDR3 of the VH region comprising the amino acid sequence SEQ ID NO: 24; and a Kabat light chain complementarity determining region-1 (VL-CDR1) amino acid sequence which is identical to the VL-CDR1 of the VL region comprising the amino acid sequence SEQ ID NO: 25; and a Kabat light chain complementarity determining region-2 (VL-CDR2) amino acid sequence which is identical to the VL-CDR2 of the VL region comprising the amino acid sequence SEQ ID NO: 25; and a Kabat light chain complementarity determining region-3 (VL-CDR3) amino acid sequence which is identical to the VL-CDR3 of the VL region comprising the amino acid sequence SEQ ID NO: 25; or
a Kabat heavy chain complementarity determining region-1 (VH-CDR1) comprising the amino acid sequence SEQ ID NO: 28; and a Kabat heavy chain complementarity determining region-2 (VH-CDR2) comprising the amino acid sequence SEQ ID NO: 29; and a Kabat heavy chain complementarity determining region-3 (VH-CDR3) comprising the amino acid sequence SEQ ID NO: 30; and a Kabat light chain complementarity determining region-1 (VL-CDR1) comprising the amino acid sequence SEQ ID NO: 31; and a Kabat light chain complementarity determining region-2 (VL-CDR2) comprising the amino acid sequence SEQ ID NO: 32; and a Kabat light chain complementarity determining region-3 (VL-CDR3) comprising the amino acid sequence SEQ ID NO: 33; or
a VH chain comprising the amino acid sequence SEQ ID NO: 24; and a VL chain comprising the amino acid sequence SEQ ID NO: 25.

According to a third embodiment (c), the present invention relates to a polynucleotide encoding an isolated IL-17B antibody, wherein said polynucleotide encodes:
a Kabat heavy chain complementarity determining region-1 (VH-CDR1) amino acid sequence which is identical to the VH-CDR1 of the VH region comprising the amino acid sequence SEQ ID NO: 40; and a Kabat heavy chain complementarity determining region-2 (VH-CDR2) amino acid sequence which is identical to the VH-CDR2 of the VH region comprising the amino acid sequence SEQ ID NO: 40; and a Kabat heavy chain complementarity determining region-3 (VH-CDR3) amino acid sequence which is identical to the VH-CDR3 of the VH region comprising the amino acid sequence SEQ ID NO: 40; and a Kabat light chain complementarity determining region-1 (VL-CDR1) amino acid sequence which is identical to the VL-CDR1 of the VL region comprising the amino acid sequence SEQ ID NO: 41; and a Kabat light chain complementarity determining region-2 (VL-CDR2) amino acid sequence which is identical to the VL-CDR2 of the VL region comprising the amino acid sequence SEQ ID NO: 41; and a Kabat light chain complementarity determining region-3 (VL-CDR3) amino acid sequence which is identical to the VL-CDR3 of the VL region comprising the amino acid sequence SEQ ID NO: 41; or
a Kabat heavy chain complementarity determining region-1 (VH-CDR1) comprising the amino acid sequence SEQ ID NO: 44; and a Kabat heavy chain complementarity determining region-2 (VH-CDR2) comprising the amino acid sequence SEQ ID NO: 45; and a Kabat heavy chain complementarity determining region-3 (VH-CDR3) comprising the amino acid sequence SEQ ID NO: 46; and a Kabat light chain complementarity determining region-1 (VL-CDR1) comprising the amino acid sequence SEQ ID NO: 47; and a Kabat light chain complementarity determining region-2 (VL-CDR2) comprising the amino acid sequence SEQ ID NO: 48; and a Kabat light chain complementarity determining region-3 (VL-CDR3) comprising the amino acid sequence SEQ ID NO: 49; or
a VH chain comprising the amino acid sequence SEQ ID NO: 40; and a VL chain comprising the amino acid sequence SEQ ID NO: 41.

According to a fourth embodiment (d), the present invention relates to a polynucleotide encoding an isolated IL-17B antibody, wherein said polynucleotide encodes:
a Kabat heavy chain complementarity determining region-1 (VH-CDR1) amino acid sequence which is identical to the VH-CDR1 of the VH region comprising the amino acid sequence SEQ ID NO: 56; and a Kabat heavy chain complementarity determining region-2 (VH-CDR2) amino acid sequence which is identical to the VH-CDR2 of the VH region comprising the amino acid sequence SEQ ID NO: 56; and a Kabat heavy chain complementarity determining region-3 (VH-CDR3) amino acid sequence which is identical to the VH-CDR3 of the VH region comprising the amino acid sequence SEQ ID NO: 56; and a Kabat light chain complementarity determining region-1 (VL-CDR1) amino acid sequence which is identical to the VL-CDR1 of the VL region comprising the amino acid sequence SEQ ID NO: 57; and a Kabat light chain complementarity determining region-2 (VL-CDR2) amino acid sequence which is identical to the VL-CDR2 of the VL region comprising the amino acid sequence SEQ ID NO: 57; and a Kabat light chain complementarity determining region-3 (VL-CDR3) amino acid sequence which is identical to the VL-CDR3 of the VL region comprising the amino acid sequence SEQ ID NO: 57; or
a Kabat heavy chain complementarity determining region-1 (VH-CDR1) comprising the amino acid sequence SEQ ID NO: 60; and a Kabat heavy chain complementarity determining region-2 (VH-CDR2) comprising the amino acid sequence SEQ ID NO: 61; and a Kabat heavy chain complementarity determining region-3 (VH-CDR3) comprising the amino acid sequence SEQ ID NO: 62; and a Kabat light chain complementarity determining region-1 (VL-CDR1) comprising the amino acid sequence SEQ ID NO: 63; and a Kabat light chain complementarity determining region-2 (VL-CDR2) comprising the amino acid sequence SEQ ID NO: 64; and a Kabat light chain complementarity determining region-3 (VL-CDR3) comprising the amino acid sequence SEQ ID NO: 65; or
a VH chain comprising the amino acid sequence SEQ ID NO: 56; and a VL chain comprising the amino acid sequence SEQ ID NO: 57.

According to another aspect, the present invention relates to a polynucleotide encoding an isolated IL-17B antibody, wherein said polynucleotide encodes a Kabat heavy chain complementarity determining region-1 (VH-CDR1) amino acid sequence which is identical, or identical except for conservative amino acid substitutions, or 80%, 85%, 90%, 91%, 92%, 93%, 94%, 95%, 96%, 97%, 98% or 99% identical to a sequence selected in the group consisting of: SEQ ID NO: 12, SEQ ID NO: 28, SEQ ID NO: 44 and SEQ ID NO: 60. In a preferred embodiment, the present invention relates to a polynucleotide which is identical or 80%, 85%, 90%, 91%, 92%, 93%, 94%, 95%, 96%, 97%, 98% or 99% identical to a sequence selected in the group consisting of: SEQ ID NO: 18, SEQ ID NO: 34, SEQ ID NO: 50 and SEQ ID NO: 66.

According to another aspect, the present invention relates to a polynucleotide encoding an isolated IL-17B antibody, wherein said polynucleotide encodes a Kabat heavy chain complementarity determining region-2 (VH-CDR2) amino acid sequence which is identical, or identical except for conservative amino acid substitutions, or 80%, 85%, 90%, 91%, 92%, 93%, 94%, 95%, 96%, 97%, 98% or 99% identical to a sequence selected in the group consisting of: SEQ ID NO: 13, SEQ ID NO: 29, SEQ ID NO: 45 and SEQ ID NO: 61. In a preferred embodiment, the present invention relates to a polynucleotide which is identical or 80%, 85%, 90%, 91%, 92%, 93%, 94%, 95%, 96%, 97%, 98% or 99% identical to a sequence selected in the group consisting of: SEQ ID NO: 19, SEQ ID NO: 35, SEQ ID NO: 51 and SEQ ID NO: 67.

According to another aspect, the present invention relates to a polynucleotide encoding an isolated IL-17B antibody, wherein said polynucleotide encodes a Kabat heavy chain complementarity determining region-3 (VH-CDR3) amino acid sequence which is identical, or identical except for conservative amino acid substitutions, or 80%, 85%, 90%, 91%, 92%, 93%, 94%, 95%, 96%, 97%, 98% or 99% identical to a sequence selected in the group consisting of: SEQ ID NO: 14, SEQ ID NO: 30, SEQ ID NO: 46 and SEQ ID NO: 62. In a preferred embodiment, the present invention relates to a polynucleotide which is identical or 80%, 85%, 90%, 91%, 92%, 93%, 94%, 95%, 96%, 97%, 98% or 99% identical to a sequence selected in the group consisting of: SEQ ID NO: 20, SEQ ID NO: 36, SEQ ID NO: 52 and SEQ ID NO: 68.

According to another aspect, the present invention relates to a polynucleotide encoding an isolated IL-17B antibody, wherein said polynucleotide encodes a Kabat light chain complementarity determining region-1 (VL-CDR1) amino acid sequence which is identical, or identical except for conservative amino acid substitutions, or 80%, 85%, 90%, 91%, 92%, 93%, 94%, 95%, 96%, 97%, 98% or 99% identical to a sequence selected in the group consisting of: SEQ ID NO: 15, SEQ ID NO: 31, SEQ ID NO: 47, and SEQ ID NO: 63. In a preferred embodiment, the present invention relates to a polynucleotide which is identical or 80%, 85%, 90%, 91%, 92%, 93%, 94%, 95%, 96%, 97%, 98% or 99% identical to a sequence selected in the group consisting of: SEQ ID NO: 21, SEQ ID NO: 37, SEQ ID NO: 53, and SEQ ID NO: 69.

According to another aspect, the present invention relates to a polynucleotide encoding an isolated IL-17B antibody, wherein said polynucleotide encodes a Kabat light chain complementarity determining region-2 (VL-CDR2) amino acid sequence which is identical, or identical except for conservative amino acid substitutions, or 80%, 85%, 90%, 91%, 92%, 93%, 94%, 95%, 96%, 97%, 98% or 99% identical to a sequence selected in the group consisting of: SEQ ID NO: 16, SEQ ID NO: 32, SEQ ID NO: 48, and SEQ ID NO: 64. In a preferred embodiment, the present invention relates to a polynucleotide which is identical or 80%, 85%, 90%, 91%, 92%, 93%, 94%, 95%, 96%, 97%, 98% or 99% identical to a sequence selected in the group consisting of: SEQ ID NO: 22, SEQ ID NO: 38, SEQ ID NO: 54, and SEQ ID NO: 70.

According to another aspect, the present invention relates to a polynucleotide encoding an isolated IL-17B antibody, wherein said polynucleotide encodes a Kabat light chain complementarity determining region-3 (VL-CDR3) amino acid sequence which is identical, or identical except for conservative amino acid substitutions, or 80%, 85%, 90%, 91%, 92%, 93%, 94%, 95%, 96%, 97%, 98% or 99% identical to a sequence selected in the group consisting of: SEQ ID NO: 17, SEQ ID NO: 33, SEQ ID NO: 49, and SEQ ID NO: 65. In a preferred embodiment, the present invention relates to a polynucleotide which is identical or 80%, 85%, 90%, 91%, 92%, 93%, 94%, 95%, 96%, 97%, 98% or 99% identical to a sequence selected in the group consisting of: SEQ ID NO: 23, SEQ ID NO: 39, SEQ ID NO: 55, and SEQ ID NO: 71.

According to another aspect, the present invention relates to a polynucleotide encoding an isolated IL-17B antibody, wherein the VH of said antibody comprises or consists of an amino acid sequence which is identical, or identical except for conservative amino acid substitutions, or 80%, 85%, 90%, 91%, 92%, 93%, 94%, 95%, 96%, 97%, 98% or 99% identical to a sequence selected in the group consisting of: SEQ ID NO: 8, SEQ ID NO: 24, SEQ ID NO: 40 and SEQ ID NO: 56. In a preferred embodiment, the present invention relates to a polynucleotide which is identical or 80%, 85%, 90%, 91%, 92%, 93%, 94%, 95%, 96%, 97%, 98% or 99% identical to a sequence selected in the group consisting of: SEQ ID NO: 10, SEQ ID NO: 26, SEQ ID NO: 42 and SEQ ID NO: 58.

According to another aspect, the present invention relates to a polynucleotide encoding an isolated IL-17B antibody, wherein the VL of said antibody comprises or consists of an amino acid sequence which is identical, or identical except for conservative amino acid substitutions, or 80%, 85%, 90%, 91%, 92%, 93%, 94%, 95%, 96%, 97%, 98% or 99% identical to a sequence selected in the group consisting of: SEQ ID NO: 9, SEQ ID NO: 25, SEQ ID NO: 41 and SEQ ID NO: 57. In a preferred embodiment, the present invention relates to a polynucleotide which is identical or 80%, 85%, 90%, 91%, 92%, 93%, 94%, 95%, 96%, 97%, 98% or 99% identical to a sequence selected in the group consisting of: SEQ ID NO: 11, SEQ ID NO: 27, SEQ ID NO: 43, and SEQ ID NO: 59.

In one embodiment, a polynucleotide of the invention comprises a nucleic acid sequence encoding light chain constant region selected from the group consisting of a human kappa constant region and a human lambda constant region. In another embodiment, a polynucleotide of the invention comprises a nucleic acid sequence encoding a heavy chain constant region or fragment thereof In a more particular embodiment, the heavy chain constant region or fragment thereof is human IgG1, IgG2, IgG3, IgG4, IgM, IgA1, IgA2, IgE or IgD.

Typically, said nucleic acid or polynucleotide is a DNA or RNA molecule, which may be included in any suitable vector, such as a plasmid, cosmid, episome, artificial chromosome, phage or a viral vector. The terms “vector”, “cloning vector” and “expression vector” mean the vehicle by which a DNA or RNA sequence (e.g. a foreign gene) can be introduced into a host cell, so as to transform the host and promote expression (e.g. transcription and translation) of the introduced sequence.

So, a further object of the invention relates to a vector comprising a nucleic acid of the invention.

Such vectors may comprise regulatory elements, such as a promoter, enhancer, terminator and the like, to cause or direct expression of said antibody upon administration to a subject. Examples of promoters and enhancers used in the expression vector for animal cell include early promoter and enhancer of SV40 (Mizukami T et al., J Biochem 101(5): 1307-10, 1987), LTR promoter and enhancer of Moloney mouse leukemia virus (Kuwana Y et al., Biochem Biophys Res Comm., 149(3): 960-8, 1987), promoter (Mason J O et al., Cell, 41(2): 479-87, 1985) and enhancer (Gillies S D et al., Cell, 33(3): 717-28, 1983) of immunoglobulin H chain and the like.

Any expression vector for animal cell can be used, so long as a gene encoding the human antibody C region can be inserted and expressed. Examples of suitable vectors include pAGE107 (Miyaji H et al., Cytotechnology, 3(2): 133-140, 1990), pAGE103 (Mizukami T et al., J Biochem 101(5): 1307-10, 1987), pHSG274 (Brady G et al., Gene, 27(2): 223-32, 1984), pKCR (O'Hare K et al, Proc. Natl. Acad. Sci USA, 78(3): 1527-31. 1981), pSG1 beta d2-4-(Miyaji H et al., Cytotechnology, 3(2): 133-140, 1990), and the like.

Other examples of plasmids include replicating plasmids comprising an origin of replication, or integrative plasmids, such as for instance pUC, pcDNA, pBR, and the like.

Other examples of viral vector include adenoviral, retroviral, herpes virus and AAV vectors. Such recombinant viruses may be produced by techniques known in the art, such as by transfecting packaging cells or by transient transfection with helper plasmids or viruses. Typical examples of virus packaging cells include PA317 cells, PsiCRIP cells, GPenv+ cells, 293 cells, etc. Detailed protocols for producing such replication-defective recombinant viruses may be found for instance in WO 95/14785, WO 96/22378, U.S. Pat. Nos. 5,882,877, 6,013,516, 4,861,719, 5,278,056 and WO 94/19478.

A further object of the present invention relates to a cell which has been transfected, infected or transformed by a nucleic acid and/or a vector according to the invention.

The term “transformation” means the introduction of a “foreign” (i.e. extrinsic or extracellular) gene, DNA or RNA sequence to a host cell, so that the host cell will express the introduced gene or sequence to produce a desired substance, typically a protein or enzyme coded by the introduced gene or sequence. A host cell that receives and expresses introduced DNA or RNA bas been “transformed”.

The nucleic acids of the invention may be used to produce an antibody of the invention in a suitable expression system. The term “expression system” means a host cell and compatible vector under suitable conditions, e.g. for the expression of a protein coded for by foreign DNA carried by the vector and introduced to the host cell.

Common expression systems include E. coli host cells and plasmid vectors, insect host cells and Baculovirus vectors, and mammalian host cells and vectors. Other examples of host cells include, without limitation, prokaryotic cells (such as bacteria) and eukaryotic cells (such as yeast cells, mammalian cells, insect cells, plant cells, etc.). Specific examples include E. coli, Kluyveromyces or Saccharomyces yeasts, mammalian cell lines (e.g., Vero cells, CHO cells, 3T3 cells, COS cells, etc.) as well as primary or established mammalian cell cultures (e.g., produced from lymphoblasts, fibroblasts, embryonic cells, epithelial cells, nervous cells, adipocytes, etc.). Examples also include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is defective (Urlaub G et al., Proc Natl Acad Sci USA, 77(7): 4216-20, 1980), rat YB2/3 HL.P2.G11.16Ag.20 cell (ATCC CRL1662, hereinafter referred to as “YB2/0 cell”), and the like.

The present invention also relates to a method of producing a recombinant host cell expressing an antibody according to the invention, said method comprising the steps of: (i) introducing in vitro or ex vivo a recombinant nucleic acid or a vector as described above into a competent host cell, (ii) culturing in vitro or ex vivo the recombinant host cell obtained and (iii), optionally, selecting the cells which express and/or secrete said antibody. Such recombinant host cells can be used for the production of antibodies of the invention.

### Methods of Producing Antibodies of the Invention

Antibodies of the invention may be produced by any technique known in the art, such as, without limitation, any chemical, biological, genetic or enzymatic technique, either alone or in combination.

Knowing the amino acid sequence of the desired sequence, one skilled in the art can readily produce said antibodies, by standard techniques for production of polypeptides. For instance, they can be synthesized using well-known solid phase method, preferably using a commercially available peptide synthesis apparatus (such as that made by Applied Biosystems, Foster City, Calif.) and following the manufacturer's instructions. Alternatively, antibodies of the invention can be synthesized by recombinant DNA techniques well-known in the art. For example, antibodies can be obtained as DNA expression products after incorporation of DNA sequences encoding the antibodies into expression vectors and introduction of such vectors into suitable eukaryotic or prokaryotic hosts that will express the desired antibodies, from which they can be later isolated using well-known techniques.

In particular, the invention further relates to a method of producing an antibody of the invention, which method comprises the steps consisting of: (i) culturing a transformed host cell according to the invention under conditions suitable to allow expression of said antibody; and (ii) recovering the expressed antibody.

In another particular embodiment, the method comprises the steps of:

(i) culturing the hybridoma under conditions suitable to allow expression of the corresponding antibody; and

(ii) recovering the expressed antibody.

Antibodies of the invention are suitably separated from the culture medium by conventional immunoglobulin purification procedures such as, for example, protein A-Sepharose, hydroxylapatite chromatography, gel electrophoresis, dialysis, or affinity chromatography.

In a particular embodiment, the human chimeric antibody of the present invention can be produced by obtaining nucleic sequences encoding VL and VH domains as previously described, constructing a human chimeric antibody expression vector by inserting them into an expression vector for animal cell having genes encoding human antibody CH and human antibody CL, and expressing the coding sequence by introducing the expression vector into an animal cell.

As the CH domain of a human chimeric antibody, it may be any region which belongs to human immunoglobulin, but those of IgG class are suitable and any one of subclasses belonging to IgG class, such as IgG 1, IgG2, IgG3 and IgG4, can also be used. Also, as the CL of a human chimeric antibody, it may be any region which belongs to Ig, and those of kappa class or lambda class can be used.

Methods for producing chimeric antibodies involve conventional recombinant DNA and gene transfection techniques are well known in the art (See Morrison S L. et al., Proc Natl Acad Sci USA, 81(21):6851-5, 1984) and patent documents U.S. Pat. Nos. 5,202,238; and 5,204, 244).

The humanized antibody of the present invention may be produced by obtaining nucleic acid sequences encoding CDR domains, as previously described, constructing a humanized antibody expression vector by inserting them into an expression vector for animal cell having genes encoding (i) a heavy chain constant region identical to that of a human antibody and (ii) a light chain constant region identical to that of a human antibody, and expressing the genes by introducing the expression vector into an animal cell.

The humanized antibody expression vector may be either of a type in which a gene encoding an antibody heavy chain and a gene encoding an antibody light chain exists on separate vectors or of a type in which both genes exist on the same vector (tandem type). In respect of easiness of construction of a humanized antibody expression vector, easiness of introduction into animal cells, and balance between the expression levels of antibody H and L chains in animal cells, humanized antibody expression vector of the tandem type is preferred (Shitara K et al., J Immunol Methods, 167(1-2): 271-8, 1994). Examples of tandem type humanized antibody expression vector include pKANTEX93 (WO 97/10354), pEE18 and the like.

Methods for producing humanized antibodies based on conventional recombinant DNA and gene transfection techniques are well known in the art (See, e.g., Riechmann L. et al., Nature, 332(6162): 323-7, 1988; Neuberger M S. et al., Nature, 312(5995): 604-8, 1985). Antibodies can be humanized using a variety of techniques known in the art including, for example, CDR-grafting (EP 239,400; PCT publication WO91/09967; U.S. Pat. Nos. 5,225,539; 5,530,101; and 5,585,089), veneering or resurfacing (EP 592,106; EP 519,596; Padlan E A, Mol Immunol., 28(4-5): 489-98, 1991); Studnicka G M et al., Protein Eng., 7(6): 805-14, 1994; Roguska M A. et al., Proc Natl Acad Sci USA, 91(3): 969-73, 1994), and chain shuffling (U.S. Pat. No. 5,565,332). The general recombinant DNA technology for preparation of such antibodies is also known (see European Patent Application EP 125023 and International Patent Application WO 96/02576).

The Fab of the present invention can be obtained by treating an antibody which specifically reacts with human IL-17B with a protease, papaine. Also, the Fab can be produced by inserting DNA encoding Fab of the antibody into a vector for prokaryotic expression system, or for eukaryotic expression system, and introducing the vector into a procaryote or eucaryote (as appropriate) to express the Fab.

The F(ab′)2 of the present invention can be obtained treating an antibody which specifically reacts with human IL-17 with a protease, pepsin. Also, the F(ab′)2 can be produced by binding Fab′ described below via a thioether bond or a disulfide bond.

The Fab′ of the present invention can be obtained treating F(ab′)2 which specifically reacts with human IL-17 with a reducing agent, dithiothreitol. Also, the Fab′ can be produced by inserting DNA encoding Fab′ fragment of the antibody into an expression vector for prokaryote, or an expression vector for eukaryote, and introducing the vector into a prokaryote or eukaryote (as appropriate) to perform its expression.

The scFv of the present invention can be produced by obtaining cDNA encoding the VH and VL domains as previously described, constructing DNA encoding scFv, inserting the DNA into an expression vector for prokaryote, or an expression vector for eukaryote, and then introducing the expression vector into a prokaryote or eukaryote (as appropriate) to express the scFv. To generate a humanized scFv fragment, a well known technology called CDR grafting may be used, which involves selecting the complementary determining regions (CDRs) from a donor scFv fragment, and grafting them onto a human scFv fragment framework of known three dimensional structure (see e. g., WO98/45322; WO 87/02671; U.S. Pat. Nos. 5,859,205; 5,585,089; 4,816,567; EP0173494).

### Modification of the Antibodies of the Invention

Amino acid sequence modification(s) of the antibodies described herein are contemplated. For example, it may be desirable to improve the binding affinity and/or other biological properties of the antibody. It is known that when a humanized antibody is produced by simply grafting only CDRs in VH and VL of an antibody derived from a non-human animal in FRs of the VH and VL of a human antibody, the antigen binding activity is reduced in comparison with that of the original antibody derived from a non-human animal. It is considered that several amino acid residues of the VH and VL of the non-human antibody, not only in CDRs but also in FRs, are directly or indirectly associated with the antigen binding activity. Hence, substitution of these amino acid residues with different amino acid residues derived from FRs of the VH and VL of the human antibody would reduce of the binding activity. In order to resolve the problem, in antibodies grafted with human CDR, attempts have to be made to identity, among amino acid sequences of the FR of the VH and VL of human antibodies, an amino acid residue which is directly associated with binding to the antibody, or which interacts with an amino acid residue of CDR, or which maintains the three-dimensional structure of the antibody and which is directly associated with binding to the antigen. The reduced antigen binding activity could be increased by replacing the identified amino acids with amino acid residues of the original antibody derived from a non-human animal.

Modifications and changes may be made in the structure of the antibodies of the present invention, and in the DNA sequences encoding them, and still obtain a functional molecule that encodes an antibody with desirable characteristics.

In making the changes in the amino sequences, the hydropathic index of amino acids may be considered. The importance of the hydropathic amino acid index in conferring interactive biologic function on a protein is generally understood in the art. It is accepted that the relative hydropathic character of the amino acid contributes to the secondary structure of the resultant protein, which in turn defines the interaction of the protein with other molecules, for example, enzymes, substrates, receptors, DNA, antibodies, antigens, and the like. Each amino acid has been assigned a hydropathic index on the basis of their hydrophobicity and charge characteristics these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophane (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

A further object of the present invention also encompasses function-conservative variants of the antibodies of the present invention.

“Function-conservative variants” are those in which a given ammo acid residue in a protein or enzyme has been changed without altering the overall conformation and function of the polypeptide, including, but not limited to, replacement of an amino acid with one having similar properties (such as, for example, polarity, hydrogen bonding potential, acidic, basic, hydrophobic, aromatic, and the like). Amino acids other than those indicated as conserved may differ in a protein so that the percent protein or amino acid sequence similarity between any two proteins of similar function may vary and may be, for example, from 70% to 99% as determined according to an alignment scheme such as by the Cluster Method, wherein similarity is based on the MEGALIGN algorithm. A “function-conservative variant” also includes a polypeptide which has at least 60% amino acid identity as determined by BLAST or FASTA algorithms, preferably at least 75%, more preferably at least 85%, still preferably at least 90%, and even more preferably at least 95%, and which has the same or substantially similar properties or functions as the native or parent protein to which it is compared.

Two amino acid sequences are “substantially homologous” or “substantially similar” or “substantially identical” when greater than 80%, preferably greater than 85%, preferably greater than 90% of the amino acids are identical, or greater than about 90%, preferably greater than 95%, are similar (functionally identical) over the whole length of the shorter sequence. Preferably, the similar or homologous sequences are identified by alignment using, for example, the GCG (Genetics Computer Group, Program Manual for the GCG Package, Version 7, Madison, Wis.) pileup program, or any of sequence comparison algorithms such as BLAST, FASTA, etc.

For example, certain amino acids may be substituted by other amino acids in a protein structure without appreciable loss of activity. Since the interactive capacity and nature of a protein define the protein's biological functional activity, certain amino acid substitutions can be made in a protein sequence, and, of course, in its DNA encoding sequence, while nevertheless obtaining a protein with like properties. It is thus contemplated that various changes may be made in the antibodies sequences of the invention, or corresponding DNA sequences which encode said antibodies, without appreciable loss of their biological activity.

It is known in the art that certain amino acids may be substituted by other amino acids having a similar hydropathic index or score and still result in a protein with similar biological activity, i.e. still obtain a biological functionally equivalent protein. As outlined above, amino acid substitutions are generally therefore based on the relative similarity of the amino acid side-chain substituents, for example, their hydrophobicity, hydrophilicity, charge, size, and the like. Exemplary substitutions which take various of the foregoing characteristics into consideration are well known to those of skill in the art and include: arginine and lysine; glutamate and aspartate; serine and threonine; glutamine and asparagine; and valine, leucine and isoleucine.

Another type of amino acid modification of the antibody of the invention may be useful for altering the original glycosylation pattern of the antibody. By «altering” is meant deleting one or more carbohydrate moieties found in the antibody, and/or adding one or more glycosylation sites that are not present in the antibody. Glycosylation of antibodies is typically N-linked. “N-linked” refers to the attachment of the carbohydrate moiety to the side chain of an asparagine residue. The tripeptide sequences asparagine-X-serine and asparagines-X-threonine, where X is any amino acid except proline, are the recognition sequences for enzymatic attachment of the carbohydrate moiety to the asparagine side chain. Thus, the presence of either of these tripeptide sequences in a polypeptide creates a potential glycosylation site. Addition of glycosylation sites to the antibody is conveniently accomplished by altering the amino acid sequence such that it contains one or more of the above-described tripeptide sequences (for N-linked glycosylation sites). Another type of covalent modification involves chemically or enzymatically coupling glycosides to the antibody. These procedures are advantageous in that they do not require production of the antibody in a host cell that has glycosylation capabilities for N-or O-linked glycosylation. Depending on the coupling mode used, the sugar(s) may be attached to (a) arginine and histidine, (b) free carboxyl groups, (c) free sulfhydryl groups such as those of cysteine, (d) free hydroxyl groups such as those of serine, threonine, orhydroxyproline, (e) aromatic residues such as those of phenylalanine, tyrosine, or tryptophan, or (f) the amide group of glutamine. For example, such methods are described in WO87/05330. Removal of any carbohydrate moieties present on the antibody may be accomplished chemically or enzymatically. Chemical deglycosylation requires exposure of the antibody to the compound trifluoromethanesulfonic acid, or an equivalent compound. This treatment results in the cleavage of most or all sugars except the linking sugar (N-acetylglucosamine or N-acetylgalactosamine), while leaving the antibody intact. Chemical deglycosylation is described by Sojahr H. et al. (1987) and by Edge, A S. et al. (Anal Biochem, 118(1): 131-7, 1981). Enzymatic cleavage of carbohydrate moieties on antibodies can be achieved by the use of a variety of endo-and exo-glycosidases as described by Thotakura, N R. et al. (Methods Enzymol., 138: 350-9, 1987).

Another type of covalent modification of the antibody comprises linking the antibody to one of a variety of non proteinaceous polymers, eg., polyethylene glycol, polypropylene glycol, or polyoxyalkylenes, in the manner set forth in U.S. Pat. Nos. 4,640,835; 4,496,689; 4,301,144; 4,670,417; 4,791,192 or 4,179,337.

It may be also desirable to modify the antibody of the invention with respect to effector function, e.g. so as to enhance antigen-dependent cell-mediated cytotoxicity (ADCC) and/or complement dependent cytotoxicity (CDC) of the antibody. This may be achieved by introducing one or more amino acid substitutions in an Fe region of the antibody. Alternatively or additionally, cysteine residue(s) may be introduced in the Fe region, thereby allowing inter-chain disulfide bond formation in this region. The homodimeric antibody thus generated may have improved internalization capability and/or increased complement-mediated cell killing and/or antibody-dependent cellular cytotoxicity (ADCC) (Caron Pc. et al., J. Exp. Med., 176(4): 1191-5, 1992; and Shopes B., J. Immunol., 148(9): 2918-22, 1992).

### Diagnostic and Therapeutic Applications of the Antibodies of the Invention

Moreover, it has been demonstrated that these antibodies act as IL-17B antagonists. Therefore, they can be used in a method for treating or preventing a disease associated with IL-17B expression or activity (“an IL-17B-mediated disease”). Said method comprises the step of administering to a subject in need thereof an antibody of the invention. The invention further provides a therapeutic method useful for treating and preventing IL-17B-mediated diseases, comprising the step of administering to a subject in need thereof an antibody of the invention.

In the context of the invention, the term “treating” or “treatment”, as used herein, means reversing, alleviating, inhibiting the progress of, or preventing the disorder or condition to which such term applies, or one or more symptoms of such disorder or condition. A “therapeutically effective amount” is intended for a minimal amount of active agent (e.g., IL-17 antibodies) which is necessary to impart therapeutic benefit to a subject. For example, a “therapeutically effective amount” to a mammal is such an amount which induces, ameliorates or otherwise causes an improvement in the pathological symptoms, disease progression or physiological conditions associated with or resistance to succumbing to a disorder.

As used herein, the term “prevention” refers to preventing the disease or condition from occurring in a subject which has not yet been diagnosed as having it.

As used herein, the term “subject” denotes a mammal, such as a rodent, a feline, a canine, and a primate. Preferably a subject according to the invention is a human.

As IL-17B-mediated diseases, immune-related and inflammatory diseases include for example: systemic lupus erythematosus, arthritis, psoriatic arthritis, rheumatoid arthritis, osteoarthritis, juvenile chronic arthritis, spondyloarthropathies, systemic sclerosis, idiopathic inflammatory myopathies, Sjogren's syndrome, systemic vasculitis, sarcoidosis, autoimmune hemolytic anemia, autoimmune thrombocytopenia, thyroiditis, diabetes mellitus, immune-mediated renal disease, demyelinating diseases of the central and peripheral nervous systems such as multiple sclerosis, idiopathic demyelinating polyneuropathy or Guillain-Barre syndrome, amyotrophic lateral sclerosis and chronic inflammatory demyelinating polyneuropathy, hepatobiliary diseases such as infectious, autoimmune chronic active hepatitis, primary biliary cirrhosis, granulomatous hepatitis, sclerosing cholangitis, inflammatory bowel disease, colitis, Crohn's disease gluten-sensitive enteropathy, and endotoxemia, autoimmune or immune-mediated skin diseases including bullous skin diseases, erythema multiforme and atopic and contact dermatitis, psoriasis, neutrophilic dermatoses, cystic fibrosis, allergic diseases such as asthma, allergic rhinitis, food hypersensitivity and urticaria, cystic fibrosis, immunologic diseases of the lung such as eosinophilic pneumonia, idiopathic pulmonary fibrosis, adult respiratory disease (ARD), acute respiratory distress syndrome (ARDS) and inflammatory lung injury such as asthma, chronic obstructive pulmonary disease (COPD), airway hyper-responsiveness, chronic bronchitis, allergic asthma and hypersensitivity pneumonitis, transplantation associated diseases including graft and organ rejection and graft -versus-host-disease, septic shock, multiple organ failure, obesity, type 2 diabetes, non alcoholic liver cirrhosis, non alcoholic liver disease, oncology (tumor angiogenesis, primary tumors and metastases; see e.g. WO 2011/141823).

Cell proliferation disorders or cancers include for example, but are not limited to: Acute Childhood Lymphoblastic Leukemia, Acute Lymphoblastic Leukemia, Acute Lymphocytic Leukemia, Acute Myeloid Leukemia, Adrenocortical Carcinoma, Adult (Primary) Hepatocellular Cancer, Adult (Primary) Liver Cancer, Adult Acute Lymphocytic Leukemia, Adult Acute Myeloid Leukemia, Adult Hodgkin's Disease, Adult Hodgkin's Lymphoma, Adult Lymphocytic Leukemia, Adult Non-Hodgkin's Lymphoma, Adult Primary Liver Cancer, Adult Soft Tissue Sarcoma, AIDS-Related Lymphoma, AIDS-Related Malignancies, Anal Cancer, Astrocytoma, Bile Duct Cancer, Bladder Cancer, Bone Cancer, Brain Stem Glioma, Brain Tumors, Breast Cancer, Cancer of the Renal Pelvis and Ureter, Central Nervous System (Primary) Lymphoma, Central Nervous System Lymphoma, Cerebellar Astrocytoma, Cerebral Astrocytoma, Cervical Cancer, Childhood (Primary) Hepatocellular Cancer, Childhood (Primary) Liver Cancer, Childhood Acute Lymphoblastic Leukemia, Childhood Acute Myeloid Leukemia, Childhood Brain Stem Glioma, Childhood Cerebellar Astrocytoma, Childhood Cerebral Astrocytoma, Childhood Extracranial Germ Cell Tumors, Childhood Hodgkin's Disease, Childhood Hodgkin's Lymphoma, Childhood Hypothalamic and Visual Pathway Glioma, Childhood Lymphoblastic Leukemia, Childhood Medulloblastoma, Childhood Non-Hodgkin's Lymphoma, Childhood Pineal and Supratentorial Primitive Neuroectodermal Tumors, Childhood Primary Liver Cancer, Childhood Rhabdomyosarcoma, Childhood Soft Tissue Sarcoma, Childhood Visual Pathway and Hypothalamic Glioma, Chronic Lymphocytic Leukemia, Chronic Myelogenous Leukemia, Colon Cancer, Cutaneous T-Cell Lymphoma, Endocrine Pancreas Islet Cell Carcinoma, Endometrial Cancer, Ependymoma, Epithelial Cancer, Esophageal Cancer, Ewing's Sarcoma and Related Tumors, Exocrine Pancreatic Cancer, Extracranial Germ Cell Tumor, Extragonadal Germ Cell Tumor, Extrahepatic Bile Duct Cancer, Eye Cancer, Female Breast Cancer, Gaucher's Disease, Gallbladder Cancer, Gastric Cancer, Gastrointestinal Carcinoid Tumor, Gastrointestinal Tumors, Germ Cell Tumors, Gestational Trophoblastic Tumor, Hairy Cell Leukemia, Head and Neck Cancer, Hepatocellular Cancer, Hodgkin's Disease, Hodgkin's Lymphoma, Hypergammaglobulinemia, Hypopharyngeal Cancer, Intestinal Cancers, Intraocular Melanoma, Islet Cell Carcinoma, Islet Cell Pancreatic Cancer, Kaposi's Sarcoma, Kidney Cancer, Laryngeal Cancer, Lip and Oral Cavity Cancer, Liver Cancer, Lung Cancer, Lymphoproliferative Disorders, Macroglobulinemia, Male Breast Cancer, Malignant Mesothelioma, Malignant Thymoma, Medulloblastoma, Melanoma, Mesothelioma, Metastatic Occult Primary Squamous Neck Cancer, Metastatic Primary Squamous Neck Cancer, Metastatic Squamous Neck Cancer, Multiple Myeloma, Multiple Myeloma/Plasma Cell Neoplasm, Myelodysplastic Syndrome, Myelogenous Leukemia, Myeloid Leukemia, Myeloproliferative Disorders, Nasal Cavity and Paranasal Sinus Cancer, Nasopharyngeal Cancer, Neuroblastoma, Non-Hodgkin's Lymphoma During Pregnancy, Nonmelanoma Skin Cancer, Non-Small Cell Lung Cancer, Occult Primary Metastatic Squamous Neck Cancer, Oropharyngeal Cancer, Osteo-/Malignant Fibrous Sarcoma, Osteosarcoma/Malignant Fibrous Histiocytoma, Osteosarcoma/Malignant Fibrous Histiocytoma of Bone, Ovarian Epithelial Cancer, Ovarian Germ Cell Tumor, Ovarian Low Malignant Potential Tumor, Pancreatic Cancer, Paraproteinemias, Purpura, Parathyroid Cancer, Penile Cancer, Pheochromocytoma, Pituitary Tumor, Plasma Cell Neoplasm/Multiple Myeloma, Primary Central Nervous System Lymphoma, Primary Liver Cancer, Prostate Cancer, Rectal Cancer, Renal Cell Cancer, Renal Pelvis and Ureter Cancer, Retinoblastoma, Rhabdomyosarcoma, Salivary Gland Cancer, Sarcoidosis Sarcomas, Sezary Syndrome, Skin Cancer, Small Cell Lung Cancer, Small Intestine Cancer, Soft Tissue Sarcoma, Squamous Neck Cancer, Stomach Cancer, Supratentorial Primitive Neuroectodermal and Pineal Tumors, T-Cell Lymphoma, Testicular Cancer, Thymoma, Thyroid Cancer, Transitional Cell Cancer of the Renal Pelvis and Ureter, Transitional Renal Pelvis and Ureter Cancer, Trophoblastic Tumors, Ureter and Renal Pelvis Cell Cancer, Urethral Cancer, Uterine Cancer, Uterine Sarcoma, Vaginal Cancer, Visual Pathway and Hypothalamic Glioma, Vulvar Cancer, Waldenstrom's Macroglobulinemia, Wilms' Tumor, and any other hyperproliferative disease, besides neoplasia, located in an organ system listed above.

Advantageously, an IL-17B-mediated disease is selected from the group consisting of autoimmune diseases (rheumatoid arthritis, Crohn's disease, multiple sclerosis, Psoriasis, Psoriatic arthritis, arthritis, uveitis, systemic Lupus erythematosus, inflammatory bowel diseases, colitis, chronic colitis, type I diabetes, diabetes), allergic diseases (type IV hypersensitivity (delayed type hypersensitivity, contact hypersensitivity), asthma, chronic obstructive pulmonary disease, atopic dermatitis, chronic allergic response, airway neutrophilia, chronic severe asthma), other immune cell mediated diseases (pulmonary fibrosis, pulmonary neutrophilia, graft versus host disease), oncology (tumor angiogenesis, primary tumors and metastases; see WO 2011/141823), osteoarthritis, vascular diseases, and atherosclerosis.

More advantageously the composition of the present invention is useful for the prevention or treatment of Rheumatoid Arthritis, Multiple Sclerosis, Systemic Lupus Erythematosus, inflammatory bowel diseases, Crohn's diseases, psoriasis, ulcerative colitis, atopic dermatitis.

More advantageously, the composition of the present invention is useful for the prevention or treatment of breast cancer, colon cancer, gastric cancer, glioma, hepatocellular carcinoma, kidney cancer, leukemia, lung cancer, lymphoma, melanoma, multiple myeloma, ovarian cancer, pancreatic cancer, prostate cancer.

Antibodies of the invention can induce cancer cell death and sensitize cells to therapeutic agents; abrogate primary tumor growth, and abrogate metastases. Hence, in one aspect, the invention is directed to a method of treating or preventing a cell proliferation disorder, said method comprising administering to said cancer cells or cells at risk for becoming cancerous an antibody of the invention. In some embodiments, the method results in targeting and/or killing the cancer cells or the cells at increased risk for becoming cancerous; increasing the effectiveness of a therapeutic agent, e.g. in treating or preventing a cell proliferation disorder; and/or preventing tumor metastasis. Advantageously, the treatment is for:
targeting and/or killing the cancer cells or the cells at increased risk for becoming cancerous;
increasing the effectiveness of a therapeutic agent, advantageously a chemotherapeutic agent or an immunotherapeutic agent; and/or
preventing or treating tumor metastasis.

In an aspect, the invention is directed to a method of increasing the effectiveness of a therapeutic agent, e.g., a chemotherapeutic agent or an immunotherapeutic agent, for killing abnormally proliferating cells in a subject having a cell proliferation disorder, said method comprising administering an amount of an antibody of the invention effective to increase the effectiveness of said therapeutic agent at a time selected from the group consisting of before, during, or after administration of said therapeutic agent.

In another aspect, the invention is directed to a method of preventing, treating or inhibiting tumor metastases or cancer invasion in a subject having a cell proliferation disorder, said method comprising administering an amount of an antibody of the invention effective to prevent or treat tumor metastases in said subject.

In a particular embodiment, the metastases originate from a primary tumor that is from a tissue or organ selected from the group consisting of breast, bladder, liver, colon, ovary, lung, kidney, cervix, stomach, intestine, prostate, esophageal, head and neck, connective tissue, and skin. In a more particular embodiment, the metastases originate from a primary tumor that is from a tissue or organ selected from the group consisting of breast, colon, lung, ovary, esophagus, head and neck, or skin (e.g., melanoma). In a more specific embodiment, the mestastasis is from a breast tumor. In another specific embodiment, the mestastasis is from a liver tumor. Non-limiting examples of types of cancer a provided elsewhere herein, and metastases could be derived from any of these cancers that has metastatic potential.

According to other embodiments, the administration of the IL-17B antibodies of the invention is combined with other treatments dedicated to the same disease. In case the further treatment also corresponds to the administration of a given molecule, said molecule can be present in the same composition as the one containing the IL-17B antibody, or can be administered separately.

In relation to cancer, the other treatment can be e.g.:
local surgery;
surgery;
radiation or radiotherapy;
chemotherapy;
immunotherapy;
targeted therapy, e.g. using BRAF/MEK inhibitors;
hormone therapy;
stem cell transplant;
precision medicine;
antitumor antibodies;
gene therapy;
vaccine;
cell therapy;
CAR (chimeric antigen receptor)—T cell therapy;
TCR (T cell receptor) therapy;
induction therapy;
consolidation therapy
maintenance therapy;
differentiating agents;
angiogenesis inhibitors.

Immunotherapy encompasses checkpoint inhibitors (CPi) e.g. targeting PD1, CTL-4 (e.g. the antibody Ipilimumab (YERVOY®)), LAG3, TIM3 and/or TIGIT.

Examples of PD1 inhibitors are Pembrolizumab, Nivolumab, BMS-936559, Cemiplimab, Avélumab, Durvalumab, Atezolizumab, Spartalizumab, or their combination.

Other immune-oncology (IO) agents include those targeting OX40, GITR, ICOS, VISTA, CD39, CD40, CD47, CD70 (e.g. anti mAbs ARGX-110 and MDX-1203), CD73 or CD137.

Vaccines are also further possible treatments, especially vaccines having PRR (Pattern Recognition Receptors such as Toll-Like Receptors or TLR) agonist properties, e.g. vaccines based on attenuated rotavirus, reovirus or on Newcastle Disease Virus (NDV).

Chemotherapeutic agents include vinca alkaloids, epipodophyllotoxins, anthracycline antibiotics, actinomycin D, plicamycin, puromycin, gramicidin D, paclitaxel (Taxol™, Bristol Myers Squibb), colchicine, cytochalasin B, emetine, maytansine, and amsacrine (or “mAMSA”). The vinca alkaloid class is described in GOODMAN AND GILMAN'S THE PHARMACOLOGICAL BASIS OF THERAPEUTICS (7th ed.), (1985), pp. 1277-1280. Exemplary of vinca alkaloids are vincristine, vinblastine, and vindesine. The epipodophyllotoxin class is described, for example, in GOODMAN AND GILMAN'S THE PHARMACOLOGICAL BASIS OF THERAPEUTICS (7th ed.), (1985), pp. 1280-1281. Exemplary of epipodophyllotoxins are etoposide, etoposide orthoquinone, and teniposide. The anthracycline antibiotic class is described in GOODMAN AND GILMAN'S THE PHARMACOLOGICAL BASIS OF THERAPEUTICS (7th ed.), (1985), pp. 1283-1285. Exemplary of anthracycline antibiotics are daunorubicin, doxorubicin, mitoxantraone, and bisanthrene. Actinomycin D, also called Dactinomycin, is described, for example, in GOODMAN AND GILMAN'S THE PHARMACOLOGICAL BASIS OF THERAPEUTICS (7th ed.), (1985), pp. 1281-1283. Plicamycin, also called mithramycin, is described in Goodmand and Gilman's The Pharmacological Basis of Therapeutics (7th ed), (1985), pp. 1287-1288. Additional chemotherapeutic agents include cisplatin (Platinol™, Bristol Myers Squibb), carboplatin (Paraplatin™, Bristol Myers Squibb), mitomycin (Mutamycin™, Bristol Myers Squibb), altretamine (Hexalen™, U.S. Bioscience, Inc.), cyclophosphamide (Cytoxan™, Bristol Myers Squibb), lomustine (CCNU) (CeeNU™ Bristol Myers Squibb), carmustine (BCNU) (BiCNU™, Bristol Myers Squibb).

Exemplary chemotherapeutic agents also include aclacinomycin A, aclarubicin, acronine, acronycine, adriamycin, aldesleukin (interleukin-2), altretamine (hexamiethylmelamine), aminoglutethimide, aminoglutethimide (cytadren), aminoimidazole carboxamide, amsacrine (m-AMSA; amsidine), anastrazole (arimidex), ancitabine, anthracyline, anthramycin, asparaginase (elspar), azacitdine, azacitidine (ladakamycin), azaguanine, azaserine, azauridine, 1,1′,1″-phosphinothioylidynetris aziridine, azirino(2′,3′:3,4)pyrrolo(1,2-a)indole-4,7-dione, BCG (theracys), BCNU, BCNU chloroethyl nitrosoureas, benzamide, 4-(bis(2-chloroethyl)amino)benzenebutanoic acid, bicalutamide, bischloroethyl nitrosourea, bleomycin (blenozane), bromodeoxyuridine, broxuridine, busulfan (myleran), carbamic acid ethyl ester, chlorambucil (leukeran), chloroethyl nitrosoureas, chorozotocin (DCNU), chromomycin A3, cis-retinoic acid, cladribine (2-chlorodeoxyadenosine; 2cda; leustatin), coformycin, cycloleucine, cyclophosphamide anhydrous, chlorambucil, cytarabine, cytarabine, cytarabine HCl (cytosar-u), 2-deoxy-2-(((methylnitrosoamino)carbonyl)amino)-D-glucose, dacarbazine, decarbazine, decarbazine (DTIC-dome), demecolcine, dexamethasone, dianhydrogalactitol, diazooxonorleucine, diethylstilbestrol, docetaxel (taxotere), eflomithine, estramustine, estramustine phosphate sodium (emcyt), ethiodized oil, ethoglucid, ethyl carbamate, ethyl methanesulfonate, fenretinide, floxuridine, floxuridine (fudr), fludarabine (fludara), fluorouracil (5-FU), fluoxymesterone (halotestin), flutamide, flutamide (eulexin), fluxuridine, gallium nitrate (granite), gemcitabine (gemzar), genistein, 2-deoxy-2-(3-methyl-3-nitrosoureido)-D-glucopyranose, goserelin (zoladex), hexestrol, hydroxyurea (hydra), idarubicin (idamycin), ifosfagemcitabine, ifosfamide (iflex), ifosfamide with mesna (MAID), interferon, interferon alfa, interferon alfa-2a, alfa-2b, alfa-n3, interleukin-2, iobenguane, iobenguane iobenguane, irinotecan (camptosar), isotretinoin (accutane), ketoconazole, 4-(bis(2-chloroethyl)amino)-L-phenylalanine, L-serine diazoacetate, lentinan, leucovorin, leuprolide acetate (LHRH-analog), levamisole (ergamisol), mannomustine, maytansine, mechlorethamine, mechlorethamine HCl (nitrogen mustard), medroxyprogesterone acetate (provera, depo provera), megestrol acetate (menace), melengestrol acetate, melphalan (alkeran), menogaril, mercaptopurin, mercaptopurine (purinethol), mercaptopurine anhydrous, MESNA, mesna (mesne), methanesulfonic acid, ethyl ester, methotrexate (mtx; methotrexate), methyl-ccnu, mimosine, misonidazole, mithramycin, mitoantrone, mitobronitol, mitoguazone, mitolactol, mitomycin (mutamycin), mitomycin C, mitotane (o,p′-DDD; lysodren), mitoxantrone HCl (novantrone), mopidamol, N,N-bis(2-chloroethyl)tetrahydro-2H-1,3,2-oxazaphosphorin-2-amine-2-oxide, N-(1-methylethyl)-4-((2-methylhydrazino)methyl)benzamide, N-methyl-bis(2-chloroethyl)amine, nicardipine, nilutamide (nilandron), nimustine, nitracrine, nitrogen mustard, nocodazole, nogalamycin, octreotide (sandostatin), pactamycin, pegaspargase (PEGx-1), pentostatin (2′-deoxycoformycin), peplomycin, peptichemio, photophoresis, picibanil, pipobroman, podofilox, podophyllotoxin, porfiromycin, prednisone, procarbazine, procarbazine HCl (matulane), prospidium, puromycin aminonucleoside, PUVA (psoralen+ultraviolet a), pyran copolymer, rapamycin, s-azacytidine, 2,4,6-tris(1-aziridinyl)-s-triazine, semustine, showdomycin, sirolimus, streptozocin (zanosar), suramin, tamoxifen citrate (nolvadex), taxon, tegafur, tenuazonic acid, TEPA, testolactone, thio-tepa, thioguanine, thiotepa (thioplex), tilorone, topotecan, tretinoin (vesanoid), triaziquone, trichodermin, triethylene glycol diglycidyl ether, triethylenemelamine, triethylenephosphoramide, triethylenethiophosphoramide, trimetrexate (neutrexin), tris(1-aziridinyl)phosphine oxide, tris(1-aziridinyl)phosphine sulfide, tris(aziridinyl)-p-benzoquinone, tris(aziridinyl)phosphine sulfide, uracil mustard, vidarabine, vidarabine phosphate, vinorelbine, vinorelbine tartrate (navelbine), (1)-mimosine, 1-(2-chloroethyl)-3-(4-methylcyclohexyl)-1-nitrosourea, (8S-cis)-10-((3-amino-2,3,6-trideoxy-alpha-L-lyxo-hexopyranosyl)oxy)-7,8,9,10-tetrahydro-6,8,11-trihydroxy-8-(hydroxyacetyl)-1-methoxy-5,12-naphthacenedione, 131-meta-iodobenzyl guanidine (I-131 MIBG), 5-(3,3-dimethyl-1-triazenyl)-1H-imidazole-4-carboxamide, 5-(bis(2-chloroethyl)amino)-2,4(1H,3H)-pyrimidinedione, 2,4,6-tris(1-aziridinyl)-s-thiazine, 2,3,5-tris(1-aziridinyl)-2,5-cyclohexadiene-1,4-dione, 2-chloro-N-(2-chloroethyl)-N-methylethanamine, N,N-bis(2-chloroethyl)tetrahydro-2H-1,3,2-oxazaphosphorin-2-amine-2-oxide, 3-deazauridine, 3-iodobenzylguanidine, 5,12-naphthacenedione, 5-azacytidine, 5-fluorouracil, (1aS,8S,8aR,8bS)-6-amino-8-(((aminocarbonyl)oxy)methyl)-1,1a, 2,8,8a, 8b -hexahydro-8a-methoxy-5-methylazirino(2′,3′:3,4)pyrrolo(1,2-a)indole-4, 7-dione, 6-azauridine, 6-mercaptopurine, 8-azaguanine, and combinations thereof.

Cytokine therapy can also be used, e.g. anti-VEGF mAbs, anti-TNFα, anti-IL-6 or anti-TGF-β. Antibodies directed to other isoforms of IL-17, e.g. IL-17A as taught in WO2013/186236, or to EGFR/HER as taught in WO2017/194554 are also of interest.

Exemplary therapeutic agents then also include, but are not limited to tyrosine kinase inhibitors (e.g., azitinib, bosutinib, canertinib, cediranib, crizotinib, dasatinib, erlotinib, gefitinib, imatinib, lapatinib, lapatinib ditosylate, neratinib, nilotinib, ruxolitinib, semaxanib, vandetanib, afatinib, TAK-285, ARRY334543, Dacomitinib, OSI-420 (Desmethyl Erlotinib), AZD8931, AEE788 (NVP-AEE788), Pelitinib (EKB-569), CUDC-101, XL647, BMS-599626 (AC480), PKC412, BD3X1382 and AP261 13) and therapeutic antibodies (e.g., abagovomab, abciximab, adalimumab, adecatumumab, alemtuzumab, altizumab, belimumab, bevacizumab, cetuximab, gemtuzumab, ibritumomab, inflilximab, nimotuzumab, panitumumab, rituximab, tositumomab, trastuzumab, zalutumumab).

Antibodies of the invention may be also used as an adjuvant of vaccine compositions.

The invention also relates to pharmaceutical composition comprising antibodies of the invention. Therefore, antibodies of the invention may be combined with pharmaceutically acceptable excipients, and optionally sustained-release matrices, such as biodegradable polymers, to form therapeutic compositions.

“Pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

The form of the pharmaceutical compositions, the route of administration, the dosage and the regimen naturally depend upon the condition to be treated, the severity of the illness, the age, weight, and sex of the patient, etc.

The pharmaceutical compositions of the invention can be formulated for a topical, oral, parenteral, intranasal, intravenous, intramuscular, subcutaneous or intraocular administration and the like.

Preferably, the pharmaceutical compositions contain vehicles which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The doses used for the administration can be adapted as a function of various parameters, and in particular as a function of the mode of administration used, of the relevant pathology, or alternatively of the desired duration of treatment.

To prepare pharmaceutical compositions, an effective amount of the antibody may be dissolved or dispersed in a pharmaceutically acceptable carrier or aqueous medium.

The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi.

Solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms.

An antibody of the invention can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride.

Prolonged absorption of the injectable compositions can be brought about by the use in the compositions of agents delaying absorption, for example, aluminium monostearate and gelatin.

Sterile injectable solutions are prepared by incorporating the active compounds in the required amount in the appropriate solvent with various of the other ingredients enumerated above, as required, followed by filtered sterilization.

Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The preparation of more, or highly concentrated solutions for direct injection is also contemplated, where the use of DMSO as solvent is envisioned to result in extremely rapid penetration, delivering high concentrations of the active agents to a small tumor area.

Upon formulation, solutions will be administered in a manner compatible with the dosage formulation and in such amount as is therapeutically effective. The formulations are easily administered in a variety of dosage forms, such as the type of injectable solutions described above, but drug release capsules and the like can also be employed.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration. In this connection, sterile aqueous media which can be employed will be known to those of skill in the art in light of the present disclosure. For example, one dosage could be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject.

The antibodies of the invention may be formulated within a therapeutic mixture to comprise about 0.0001 to 1.0 milligrams, or about 0.001 to 0.1 milligrams, or about 0.1 to 1.0 or even about 10 milligrams per dose or so. Multiple doses can also be administered.

In addition to the compounds formulated for parenteral administration, such as intravenous or intramuscular injection, other pharmaceutically acceptable forms include, e.g. tablets or other solids for oral administration; time release capsules; and any other form currently used.

In certain embodiments, the use of liposomes and/or nanoparticles is contemplated for the introduction of antibodies into host cells. The formation and use of liposomes and/or nanoparticles are known to those of skill in the art.

Nanocapsules can generally entrap compounds in a stable and reproducible way. To avoid side effects due to intracellular polymeric overloading, such ultrafine particles (sized around 0.1 μm) are generally designed using polymers able to be degraded in vivo. Biodegradable polyalkyl-cyanoacrylate nanoparticles that meet these requirements are contemplated for use in the present invention, and such particles may be are easily made.

Liposomes are formed from phospholipids that are dispersed in an aqueous medium and spontaneously form multilamellar concentric bilayer vesicles (also termed multilamellar vesicles (MLVs)). MLVs generally have diameters of from 25 nm to 4 μm. Sonication of MLVs results in the formation of small unilamellar vesicles (SUVs) with diameters in the range of 200 to 500 Å, containing an aqueous solution in the core. The physical characteristics of liposomes depend on pH, ionic strength and the presence of divalent cations.

The invention also provides kits comprising at least one antibody of the invention. Kits containing antibodies of the invention find use in diagnostic and therapeutic assays.

### Diagnostics

Advantageously, The invention further provides a diagnostic method useful during diagnosis of IL-17B-mediated diseases such as neoplastic disorders or inflammatory diseases, including solid tumors, which involves measuring the expression level of IL-17B protein or transcript in tissue or other cells or body fluid from an individual and comparing the measured expression level with a standard IL-17B expression level in normal tissue or body fluid, whereby an increase in the expression level compared to the standard is indicative of a disorder.

The anti-IL-17B antibodies of the invention and antigen-binding fragments, variants, and derivatives thereof, can be used to assay IL-17B protein levels in a biological sample using classical immunohistological methods known to those of skill in the art (e.g., see Jalkanen, et al., J. Cell. Biol. 101:976-985 (1985); Jalkanen et al., J. Cell Biol. 105:3087-3096 (1987)). Other antibody-based methods useful for detecting IL-17B protein expression include immunoassays, such as the enzyme linked immunosorbent assay (ELISA), immunoprecipitation, or Western blotting. Suitable assays are described in more detail elsewhere herein.

By “assaying the expression level of IL-17B polypeptide” is intended qualitatively or quantitatively measuring or estimating the level of IL-17B polypeptide in a first biological sample either directly (e.g., by determining or estimating absolute protein level) or relatively (e.g., by comparing to the disease associated polypeptide level in a second biological sample). Preferably, IL-17B polypeptide expression level in the first biological sample is measured or estimated and compared to a standard IL-17B polypeptide level, the standard being taken from a second biological sample obtained from an individual not having the disorder or being determined by averaging levels from a population of individuals not having the disorder. As will be appreciated in the art, once the “standard” IL-17B polypeptide level is known, it can be used repeatedly as a standard for comparison.

By “biological sample” is intended any biological sample obtained from an individual, cell line, tissue culture, or other source of cells potentially expressing IL-17B. Methods for obtaining tissue biopsies and body fluids from mammals are well known in the art.

The anti-IL-17B antibodies for use in the diagnostic methods described above in this section are intended to include those anti-IL-17B antibodies, or fragments, variants, or derivatives that are described in detail elsewhere herein as if they were separately listed in this section.

### Immunoassays

Advantageously, anti-IL-17B antibodies, or antigen-binding fragments, variants, or derivatives thereof of the invention may be assayed for immunospecific binding by any method known in the art. The immunoassays that can be used include but are not limited to competitive and non-competitive assay systems using techniques such as Western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds, (1994) CURRENT PROTOCOLS IN MOLECULAR BIOLOGY (John Wiley & Sons, Inc., NY) Vol. 1, which is incorporated by reference herein in its entirety). Exemplary immunoassays are described briefly below (but are not intended by way of limitation).

Immunoprecipitation protocols generally comprise lysing a population of cells in a lysis buffer such as RIPA buffer (1% NP-40 or Triton X-100, 1% sodium deoxycholate, 0.1% SDS, 0.15 M NaCl, 0.01 M sodium phosphate at pH 7.2, 1% Trasylol) supplemented with protein phosphatase and/or protease inhibitors (e.g., EDTA, PMSF, aprotinin, sodium vanadate), adding the antibody of interest to the cell lysate, incubating for a period of time (e.g., 1-4 hours) at 4° C., adding protein A and/or protein G sepharose beads to the cell lysate, incubating for about an hour or more at 4° C., washing the beads in lysis buffer and resuspending the beads in SDS/sample buffer. The ability of the antibody of interest to immunoprecipitate a particular antigen can be assessed by, e.g., western blot analysis. One of skill in the art would be knowledgeable as to the parameters that can be modified to increase the binding of the antibody to an antigen and decrease the background (e.g., pre-clearing the cell lysate with sepharose beads). For further discussion regarding immunoprecipitation protocols see, e.g., Ausubel et al., eds, (1994) Current Protocols in Molecular Biology (John Wiley & Sons, Inc., NY) Vol. 1 at 10.16.1.

Western blot analysis generally comprises preparing protein samples, electrophoresis of the protein samples in a polyacrylamide gel (e.g., 8%-20% SDS-PAGE depending on the molecular weight of the antigen), transferring the protein sample from the polyacrylamide gel to a membrane such as nitrocellulose, PVDF or nylon, blocking the membrane in blocking solution (e.g., PBS with 3% BSA or non-fat milk), washing the membrane in washing buffer (e.g., PBS-Tween 20), blocking the membrane with primary antibody (the antibody of interest) diluted in blocking buffer, washing the membrane in washing buffer, blocking the membrane with a secondary antibody (which recognizes the primary antibody, e.g., an anti-human antibody) conjugated to an enzymatic substrate (e.g., horseradish peroxidase or alkaline phosphatase) or radioactive molecule (e.g., ³²P or ¹²⁵I) diluted in blocking buffer, washing the membrane in wash buffer, and detecting the presence of the antigen. One of skill in the art would be knowledgeable as to the parameters that can be modified to increase the signal detected and to reduce the background noise. For further discussion regarding western blot protocols see, e.g., Ausubel et al., eds, (1994) Current Protocols in Molecular Biology (John Wiley & Sons, Inc., NY) Vol. 1 at 10.8.1.

ELISAs comprise preparing antigen, coating the well of a 96-well microtiter plate with the antigen, adding the antibody of interest conjugated to a detectable compound such as an enzymatic substrate (e.g., horseradish peroxidase or alkaline phosphatase) to the well and incubating for a period of time, and detecting the presence of the antigen. In ELISAs the antibody of interest does not have to be conjugated to a detectable compound; instead, a second antibody (which recognizes the antibody of interest) conjugated to a detectable compound may be added to the well. Further, instead of coating the well with the antigen, the antibody may be coated to the well. In this case, a second antibody conjugated to a detectable compound may be added following the addition of the antigen of interest to the coated well. One of skill in the art would be knowledgeable as to the parameters that can be modified to increase the signal detected as well as other variations of ELISAs known in the art. For further discussion regarding ELISAs see, e.g., Ausubel et al., eds, (1994) Current Protocols in Molecular Biology (John Wiley & Sons, Inc., NY) Vol. 1 at 11.2.1.

The binding affinity of an antibody to an antigen and the off-rate of an antibody-antigen interaction can be determined by competitive binding assays. One example of a competitive binding assay is a radioimmunoassay comprising the incubation of labeled antigen (e.g., ³H or ¹²⁵I) with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest is conjugated to a labeled compound (e.g., ³H or ¹²⁵I) in the presence of increasing amounts of an unlabeled second antibody.

Anti-IL-17B antibodies, or antigen-binding fragments, variants, or derivatives thereof of the invention, additionally, can be employed histologically, as in immunofluorescence, immunoelectron microscopy or non-immunological assays, for in situ detection of IL-17B protein or conserved variants or peptide fragments thereof. In situ detection may be accomplished by removing a histological specimen from a patient, and applying thereto a labeled anti-IL-17B antibody, or antigen-binding fragment, variant, or derivative thereof, preferably applied by overlaying the labeled antibody (or fragment) onto a biological sample. Through the use of such a procedure, it is possible to determine not only the presence of IL-17B protein, or conserved variants or peptide fragments, but also its distribution in the examined tissue. Using the present invention, those of ordinary skill will readily perceive that any of a wide variety of histological methods (such as staining procedures) can be modified in order to achieve such in situ detection.

Immunoassays and non-immunoassays for IL-17B gene products or conserved variants or peptide fragments thereof will typically comprise incubating a sample, such as a biological fluid, a tissue extract, freshly harvested cells, or lysates of cells which have been incubated in cell culture, in the presence of a detectably labeled antibody capable of binding to IL-17B or conserved variants or peptide fragments thereof, and detecting the bound antibody by any of a number of techniques well known in the art.

The biological sample may be brought in contact with and immobilized onto a solid phase support or carrier such as nitrocellulose, or other solid support which is capable of immobilizing cells, cell particles or soluble proteins. The support may then be washed with suitable buffers followed by treatment with the detectably labeled anti-IL-17B antibody, or antigen-binding fragment, variant, or derivative thereof. The solid phase support may then be washed with the buffer a second time to remove unbound antibody. Optionally the antibody is subsequently labeled. The amount of bound label on solid support may then be detected by conventional means.

By “solid phase support or carrier” is intended any support capable of binding an antigen or an antibody. Well-known supports or carriers include glass, polystyrene, polypropylene, polyethylene, dextran, nylon, amylases, natural and modified celluloses, polyacrylamides, gabbros, and magnetite. The nature of the carrier can be either soluble to some extent or insoluble for the purposes of the present invention. The support material may have virtually any possible structural configuration so long as the coupled molecule is capable of binding to an antigen or antibody. Thus, the support configuration may be spherical, as in a bead, or cylindrical, as in the inside surface of a test tube, or the external surface of a rod. Alternatively, the surface may be flat such as a sheet, test strip, etc. Preferred supports include polystyrene beads. Those skilled in the art will know many other suitable carriers for binding antibody or antigen, or will be able to ascertain the same by use of routine experimentation.

The binding activity of a given lot of anti-IL-17B antibody, or antigen-binding fragment, variant, or derivative thereof may be determined according to well known methods. Those skilled in the art will be able to determine operative and optimal assay conditions for each determination by employing routine experimentation.

There are a variety of methods available for measuring the affinity of an antibody-antigen interaction, but relatively few for determining rate constants. Most of the methods rely on either labeling antibody or antigen, which inevitably complicates routine measurements and introduces uncertainties in the measured quantities.

Surface plasmon reasonance (SPR) as performed on BIACORE® offers a number of advantages over conventional methods of measuring the affinity of antibody-antigen interactions: (i) no requirement to label either antibody or antigen; (ii) antibodies do not need to be purified in advance, cell culture supernatant can be used directly; (iii) real-time measurements, allowing rapid semi-quantitative comparison of different monoclonal antibody interactions, are enabled and are sufficient for many evaluation purposes; (iv) biospecific surface can be regenerated so that a series of different monoclonal antibodies can easily be compared under identical conditions; (v) analytical procedures are fully automated, and extensive series of measurements can be performed without user intervention. BIAapplications Handbook, version AB (reprinted 1998), BIACORE® code No. BR-1001-86; BIAtechnology Handbook, version AB (reprinted 1998), BIACORE® code No. BR-1001-84. SPR based binding studies require that one member of a binding pair be immobilized on a sensor surface. The binding partner immobilized is referred to as the ligand. The binding partner in solution is referred to as the analyte. In some cases, the ligand is attached indirectly to the surface through binding to another immobilized molecule, which is referred as the capturing molecule. SPR response reflects a change in mass concentration at the detector surface as analytes bind or dissociate.

Based on SPR, real-time BIACORE® measurements monitor interactions directly as they happen. The technique is well suited to determination of kinetic parameters. Comparative affinity ranking is simple to perform, and both kinetic and affinity constants can be derived from the sensorgram data.

When analyte is injected in a discrete pulse across a ligand surface, the resulting sensorgram can be divided into three essential phases: (i) Association of analyte with ligand during sample injection; (ii) Equilibrium or steady state during sample injection, where the rate of analyte binding is balanced by dissociation from the complex; (iii) Dissociation of analyte from the surface during buffer flow.

The association and dissociation phases provide information on the kinetics of analyte-ligand interaction (kₐ and k_{d}, the rates of complex formation and dissociation, k_{d}/kₐ=k_{D}). The equilibrium phase provides information on the affinity of the analyte-ligand interaction (K_{D}).

BIAevaluation software provides comprehensive facilities for curve fitting using both numerical integration and global fitting algorithms. With suitable analysis of the data, separate rate and affinity constants for interaction can be obtained from simple BIACORE® investigations. The range of affinities measurable by this technique is very broad ranging from mM to pM.

Epitope specificity is an important characteristic of a monoclonal antibody. Epitope mapping with BIACORE®, in contrast to conventional techniques using radioimmunoassay, ELISA or other surface adsorption methods, does not require labeling or purified antibodies, and allows multi-site specificity tests using a sequence of several monoclonal antibodies. Additionally, large numbers of analyses can be processed automatically.

Pair-wise binding experiments test the ability of two MAbs to bind simultaneously to the same antigen. MAbs directed against separate epitopes will bind independently, whereas MAbs directed against identical or closely related epitopes will interfere with each other's binding. These binding experiments with BIACORE® are straightforward to carry out.

For example, one can use a capture molecule to bind the first Mab, followed by addition of antigen and second MAb sequentially. The sensorgrams will reveal: (1) how much of the antigen binds to first Mab, (2) to what extent the second MAb binds to the surface-attached antigen, (3) if the second MAb does not bind, whether reversing the order of the pair-wise test alters the results.

Peptide inhibition is another technique used for epitope mapping. This method can complement pair-wise antibody binding studies, and can relate functional epitopes to structural features when the primary sequence of the antigen is known. Peptides or antigen fragments are tested for inhibition of binding of different MAbs to immobilized antigen. Peptides which interfere with binding of a given MAb are assumed to be structurally related to the epitope defined by that MAb.

The anti-IL-17B antibodies for use in the immunoassays described above in this section are intended to include those anti-IL-17B antibodies, or fragments, variants, or derivatives that are described in detail elsewhere herein as if they were separately listed in this section.

The practice of the present invention will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Sambrook et al., ed. (1989) Molecular Cloning A Laboratory Manual (2nd ed.; Cold Spring Harbor Laboratory Press); Sambrook et al., ed. (1992) Molecular Cloning: A Laboratory Manual, (Cold Springs Harbor Laboratory, NY); D. N. Glover ed., (1985) DNA Cloning, Volumes I and II; Gait, ed. (1984) Oligonucleotide Synthesis; Mullis et al. U.S. Pat. No. 4,683,195; Hames and Higgins, eds. (1984) Nucleic Acid Hybridization; Hames and Higgins, eds. (1984) Transcription And Translation; Freshney (1987) Culture Of Animal Cells (Alan R. Liss, Inc.); Immobilized Cells And Enzymes (IRL Press) (1986); Perbal (1984) A Practical Guide To Molecular Cloning; the treatise, Methods In Enzymology (Academic Press, Inc., N.Y.); Miller and Calos eds. (1987) Gene Transfer Vectors For Mammalian Cells, (Cold Spring Harbor Laboratory); Wu et al., eds., Methods In Enzymology, Vols. 154 and 155; Mayer and Walker, eds. (1987) Immunochemical Methods In Cell And Molecular Biology (Academic Press, London); Weir and Blackwell, eds., (1986) Handbook Of Experimental Immunology, Volumes I-IV; Manipulating the Mouse Embryo, Cold Spring Harbor Laboratory Press, Cold Spring Harbor, N.Y., (1986); and in Ausubel et al. (1989) Current Protocols in Molecular Biology (John Wiley and Sons, Baltimore, Md.).

General principles of antibody engineering are set forth in Borrebaeck, ed. (1995) Antibody Engineering (2nd ed.; Oxford Univ. Press). General principles of protein engineering are set forth in Rickwood et al., eds. (1995) Protein Engineering, A Practical Approach (IRL Press at Oxford Univ. Press, Oxford, Eng.). General principles of antibodies and antibody-hapten binding are set forth in: Nisonoff (1984) Molecular Immunology (2nd ed.; Sinauer Associates, Sunderland, Mass.); and Steward (1984) Antibodies, Their Structure and Function (Chapman and Hall, New York, N.Y.). Additionally, standard methods in immunology known in the art and not specifically described are generally followed as in Current Protocols in Immunology, John Wiley & Sons, New York; Stites et al., eds. (1994) Basic and Clinical Immunology (8th ed; Appleton & Lange, Norwalk, Conn.) and Mishell and Shiigi (eds) (1980) Selected Methods in Cellular Immunology (W.H. Freeman and Co., NY).

Standard reference works setting forth general principles of immunology include Current Protocols in Immunology, John Wiley & Sons, New York; Klein (1982) J., Immunology: The Science of Self-Nonself Discrimination (John Wiley & Sons, NY); Kennett et al., eds. (1980) Monoclonal Antibodies, Hybridoma: A New Dimension in Biological Analyses (Plenum Press, NY); Campbell (1984) “Monoclonal Antibody Technology” in Laboratory Techniques in Biochemistry and Molecular Biology, ed. Burden et al., (Elsevere, Amsterdam); Goldsby et al., eds. (2000) Kuby Immunology (4th ed.; H. Freemand & Co.); Roitt et al. (2001) Immunology (6th ed.; London: Mosby); Abbas et al. (2005) Cellular and Molecular Immunology (5th ed.; Elsevier Health Sciences Division); Kontermann and Dubel (2001) Antibody Engineering (Springer Verlan); Sambrook and Russell (2001) Molecular Cloning: A Laboratory Manual (Cold Spring Harbor Press); Lewin (2003) Genes VIII (Prentice Hall 2003); Harlow and Lane (1988) Antibodies: A Laboratory Manual (Cold Spring Harbor Press); Dieffenbach and Dveksler (2003) PCR Primer (Cold Spring Harbor Press).

The invention will further be illustrated in view of the following examples, which are not intended to be limiting.

### BRIEF DESCRIPTION OF THE FIGURES

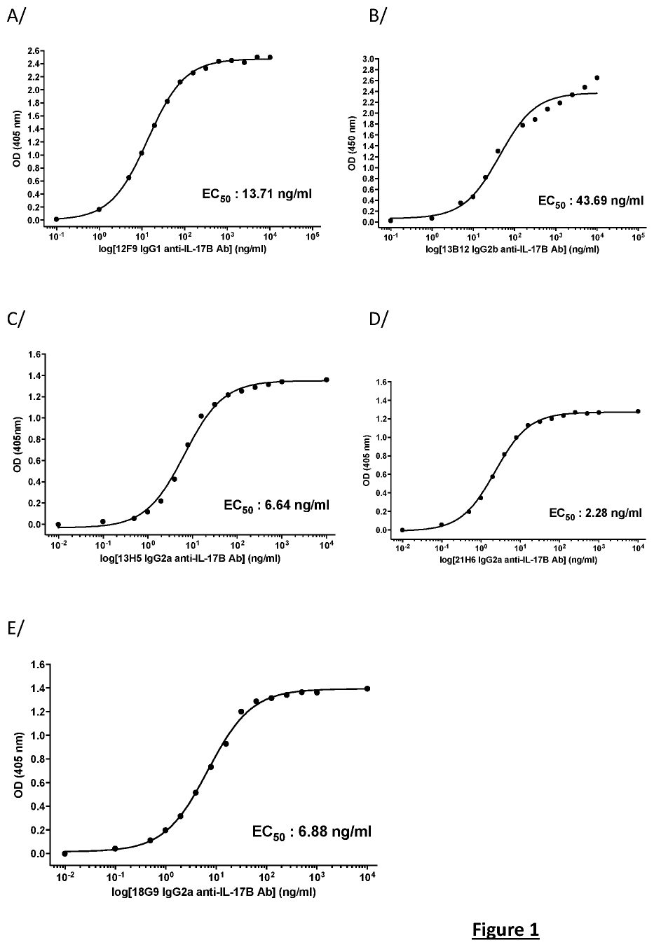

FIG. 1: Binding of various anti-IL-17B antibodies (mouse monoclonal antibodies directed against human IL-17B) on human IL-17B:
(A) Antibody 12F9
(B) Antibody 13B12
(C) Antibody 13H5
(D) Antibody 21H6
(E) Antibody 18G9

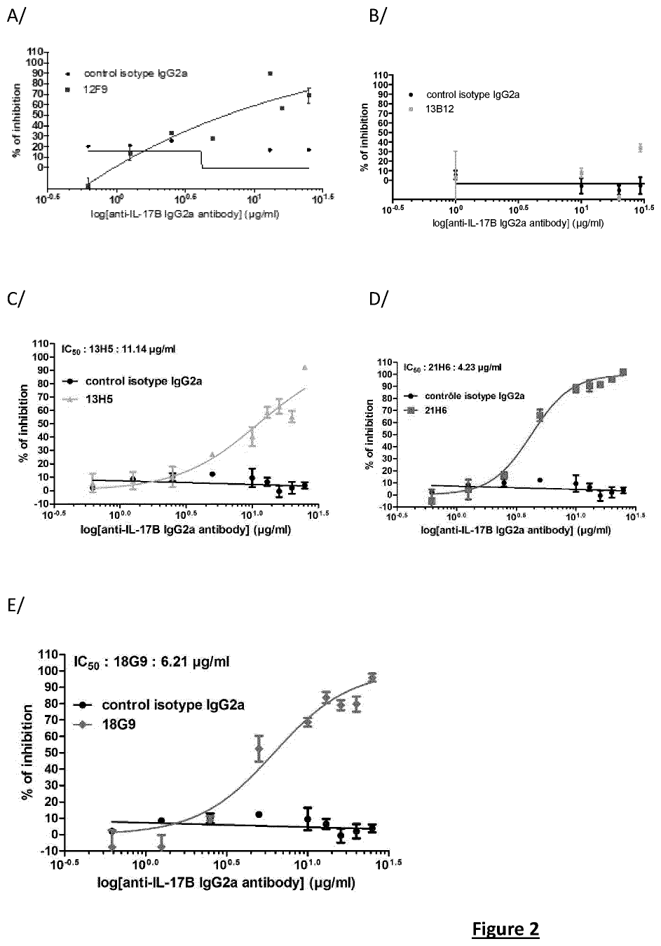

FIG. 2: Human IL-17B induced IL-8 secretion by HepG2 cells in the presence of various anti-IL-17B antibodies (mouse monoclonal antibodies directed against human IL-17B). The % of inhibition is shown for
(A) Antibody 12F9
(B) Antibody 13B12
(C) Antibody 13H5
(D) Antibody 21H6
(E) Antibody 18G9
   as well as for the corresponding control antibody (isotype IgG2a).

### EXAMPLES

### A—Experimental Procedures

I—Binding on Human IL-17B

The following protocol was implemented:
Dilute human IL-17B (R&D Systems, ref : 1248-IB/CF) at 500 ng/ml in PBS;
Immediately coat a Maxisorp 96-well plate;
Seal the plate and incubate overnight at 4° C.;
Aspirate each well and wash with wash buffer (PBS-Tween 0.05%); repeating the process two times;
Block plate by adding 200 μl of PBS-BSA 1% for 2 hours at room temperature (RT);
Aspirate each well and wash with wash buffer (PBS-Tween 0.05%); repeating the process two times;
Add 100 μl of antibodies in dilution from 100 μg/ml to 0.0001 μg/ml in PBS-Tween 0.05%;
Incubate for 2 hours at RT;
Aspirate each well and wash with wash buffer (PBS-Tween 0.05%); repeating the process two times;
Incubate with 100 μl of Goat anti-mouse HRP (Thermo Scientific, ref: 31437) diluted in PBS-BSA 0.5% for 1 hour at RT;
Aspirate each well and wash with wash buffer; repeating the process two times;
Incubate 15 min at RT with 50 μl of ABTS substrate (Sigma-Aldrich, ref: A3219-100 ML)
Measure DO (Optical Density) at 405 nm

II—Human IL-17B Induced IL-8 Secretion by HepG2 Cells

The following protocol was implemented:

At Day 1:
Prepare antibodies in dilution (4.4×) in MEM 2% SVF;
Prepare human IL-17B (R&D Systems, ref: 1248-IB/CF) at 8.8 μg/ml (4.4×) in MEM 2% SVF;
Mix 25 μl of diluted antibodies with 25 μl of hIL-17B and incubate the mix during 30 min at room temperature (RT);
Prepare polymyxin B (Sigma-Aldrich, ref : P4932-5MU) at 110 μg/ml in MEM 2% SVF;
Prepare HepG2 cells suspension at 400.000 cells/ml after trypsinization;
Mix in 96-well plate 50 μl of the mix antibody/hIL-17B, 50 μl of the cell suspension and 10 μl of polymyxin;
Place the plate for 24 hours at 37° C., 5% CO₂;
Prepare αIL-8 antibody solution (R&D Systems, ref: MAB208) at 4 μg/ml in PBS and coat a Maxisorp 96-well plate;
Seal the plate and incubate overnight at 4° C.;

At Day 2:
Aspirate each well and wash with wash buffer (PBS-Tween 0.05%); repeating the process two times;
Block plate by adding 200 μl of PBS-BSA 1% for 2 hours at room temperature (RT);
Aspirate each well;
Add 50 μl of PBS-BSA 0.5% and 50 μl of standard or HepG2 pure supernatants;
Incubate for 2 hours at RT;
Aspirate each well and wash with wash buffer (PBS-Tween 0.05%); repeating the process two times;
Incubate with 100 μl of biotinylated αIL-8 diluted (R&D Systems, ref: BAB208) in PBS-BSA 0.5% for 2 hours at RT;
Aspirate each well and wash with wash buffer; repeating the process two times;
Incubate with 100 μl of SA-HRP (R&D Systems, ref: DY998) in PBS-BSA 0.5% for 30 min at RT;
Aspirate each well and wash with wash buffer; repeating the process two times;
Incubate with 100 μl of 1:1 color reagent A+color reagent B (R&D Systems, ref: DY999) for 10 min at RT;
Add 50 μl of Stop solution (B (R&D Systems, ref: DY994);
D.O at 450 nm with correction at 540 nm.

### B—Results

I/Tested Antibodies

| | Epitope | Sequences | |
|---|---|---|---|
| Name | recognized | VH domain | VL domain |
| 12F9 | QVPLDLVSR | VH: SEQ ID NO: 8 | VL: SEQ ID NO: 9 |
| | (SEQ ID NO: 3) | CDR1: SEQ ID NO: 12 = | CDR1: SEQ ID NO: 15 = |
| | | DYFIN | RSSQSIVHSNGNTYLE |
| | | CDR2: SEQ ID NO: 13 = | CDR2: SEQ ID NO: 16 = |
| | | WIFPGSGSTYYHEKFKG | KVSNRFS |
| | | CDR3: SEQ ID NO: 14 = | CDR3: SEQ ID NO: 17 |
| | | TLYGNWYFDV | FQGSHVPYT |
| 13B12 | KPYARMEEY | Not Determined | Not Determined |
| | (SEQ ID NO: 7) + | | |
| | SQVPVRRR | | |
| | (SEQ ID NO: 4) + | | |
| | PPPPRTG | | |
| | (SEQ ID NO: 5 | | |
| | truncated) | | |
| 13H5 | QVPLDLVSR | VH: SEQ ID NO: 24 | VL: SEQ ID NO: 25 |
| | (SEQ ID NO: 3) + | CDR1: SEQ ID NO: 28 = | CDR1: SEQ ID NO: 31 = |
| | SQVPVRRR | TFGMGVG | SASSSVSYMY |
| | (SEQ ID NO: 4) + | CDR2: SEQ ID NO: 29 = | CDR2: SEQ ID NO: 32 = |
| | PPPPRTGPCRQ | HIWWDDDKYYNPALKS | DTSNLAS |
| | (SEQ ID NO: 5) | CDR3: SEQ ID NO: 30 = | CDR3: SEQ ID NO: 33 = |
| | | MNDGYLYY | QQWSSYPFT |
| 21H6 | QVPLDLVSR | VH: SEQ ID NO: 40 | VL: SEQ ID NO: 41 |
| | (SEQ ID NO: 3) + | CDR1: SEQ ID NO: 44 = | CDR1: SEQ ID NO: 47 = |
| | SQVPVRRR | TFGMGVG | RASQNISDYLH |
| | (SEQ ID NO: 4) + | CDR2: SEQ ID NO: 45 = | CDR2: SEQ ID NO: 48 = |
| | PPPPRTGPCRQ | HIWWDDDKYYNPALKG | YTSQSIS |
| | (SEQ ID NO: 5) | CDR3: SEQ ID NO: 46 = | CDR3: SEQ ID NO: 49 = |
| | | IEDALDY | QNGHSFPFT |
| 18G9 | QVPLDLVSR | VH: SEQ ID NO: 56 | VL: SEQ ID NO: 57 |
| | (SEQ ID NO: 3) + | CDR1: SEQ ID NO: 60 = | CDR1: SEQ ID NO: 63 = |
| | SQVPVRRR | TSGMGVG | KASQSVDYDGDSYMN |
| | (SEQ ID NO: 4) + | CDR2: SEQ ID NO: 61 = | CDR2: SEQ ID NO: 64 = |
| | PPPPRTGPCRQ | HIWWDDDKYYNPSLKS | AASNLES |
| | (SEQ ID NO: 5) + | CDR3: SEQ ID NO: 62 = | CDR3: SEQ ID NO: 65 = |
| | CEVNLQLWMS | RTQGYFDY | QQSNEDPLT |
| | (SEQ ID NO: 6) | | |

II/Binding

The binding data are shown on FIG. 1. Despite the fact that they recognize different sequences on the human IL-17B cytokine, they are all able to bind IL17B with EC₅₀ values ranging from 2.28 ng/ml to 43.69 ng/ml.

III/Neutralizing Activity

The data reflecting the antagonist activity of each of said antibodies are shown on FIG. 2.

Interestingly, antibody 13B12 which is not able to bind the sequence QVPLDLVSR (SEQ ID NO: 3 corresponding to residues 44 to 52 of the human IL-17B of sequence SEQ ID NO: 1), but sequence KPYARMEEY (SEQ ID NO: 7) instead, displays no inhibitory effect. This reveals that the sequence QVPLDLVSR would be of high relevance for the neutralizing activity.

This is confirmed by the data obtained with antibody 12F9 which is merely able to bind said sequence on the human IL-17B and which shows a weak but inhibitory effect.

Lower EC₅₀ values are observed with the 3 other antibodies which are further able to bind sequences SQVPVRRR (SEQ ID NO: 4 corresponding to residues 145 to 152 of the human IL-17B) and PPPPRTGPCRQ (SEQ ID NO: 5 corresponding to residues 155 to 165 of the human IL-17B) (and further CEVNLQLWMS (SEQ ID NO: 6) for 18G9). It is to be noted that based on in silico modelization, region 145-165, encompassing SEQ ID NO: 4 and SEQ ID NO: 5, could be involved in the interaction between I1-17B and its receptor.

In conclusion, the results show that the sequence QVPLDLVSR (SEQ ID NO: 3) of human IL-17B is a key sequence for its activity and then a target for antagonist antibodies. Further targeting region 145-165 would also be important for getting a better inhibition.

### REFERENCES

Gaffen, S. L. (2009) “Structure and signalling in the IL-17 receptor family” Nature reviews. Immunology 9(8): 556-567
Iwakura, Y. et al. (2011) “Functional specialization of interleukin-17 family members” Immunity 34(2): 149-162
Lee, J. et al. (2001) “IL-17E, a novel proinflammatory ligand for the IL-17 receptor homolog IL-17Rh1” J Biol Chem 276(2): 1660-1664
Ramirez-Carrozzi, V. et al. (2011) “IL-17C regulates the innate immune function of epithelial cells in an autocrine manner” Nat Immunol 12(12): 1159-1166
Stamp, L. K. et al. (2008) “Different T cell subsets in the nodule and synovial membrane: absence of interleukin-17A in rheumatoid nodules” Arthritis Rheum 58(6): 1601-1608
Starnes, T. et al. (2002) “Cutting edge: IL-17D, a novel member of the IL-17 family, stimulates cytokine production and inhibits hemopoiesis” J Immunol 169(2): 642-646
Wong, C. K. et al. (2005) “Interleukin-25-induced chemokines and interleukin-6 release from eosinophils is mediated by p38 mitogen-activated protein kinase, c-Jun N-terminal kinase, and nuclear factor-kappaB” American journal of respiratory cell and molecular biology 33(2): 186-194
Yamaguchi (2007) “IL-17B and IL-17C are associated with TNF-α production and contribute to the exacerbation of inflammatory arthritis” J. Immunol 179: 7128-7136

1. An isolated IL-17B antibody which binds the sequence of the human IL-17B as set forth in SEQ ID NO: 3.

2. The antibody of claim 1 which further binds a sequence selected from the group consisting of SEQ NOs: 4 to 6.

3. The antibody of claim 1 which comprises:
a Kabat heavy chain complementarity determining region-1 (VH-CDR1) amino acid sequence which is identical to the VH-CDR1 of the VH region comprising the amino acid sequence selected from the group consisting of:
(a) SEQ ID NO: 8,
(b) SEQ ID NO:24,
(c) SEQ ID NO:40, and
(d) SEQ ID NO: 56, and
a Kabat heavy chain complementarity determining region-2 (VH-CDR2) amino acid sequence which is identical to the VH-CDR2 of the VH region comprising the amino acid sequence selected from the group consisting of:
(a) SEQ ID NO: 8,
(b) SEQ ID NO:24,
(c) SEQ ID NO:40, and
(d) SEQ ID NO: 56, and
a Kabat heavy chain complementarity determining region-3 (VH-CDR3) amino acid sequence which is identical to the VH-CDR3 of the VH region comprising the amino acid sequence selected from the group consisting of:
(a) SEQ ID NO: 8,
(b) SEQ ID NO:24,
(c) SEQ ID NO:40, and
(d) SEQ ID NO: 56, and
a Kabat light chain complementarity determining region-1 (VL-CDR1) amino acid sequence which is identical to the VL-CDR1 of the VL region comprising the amino acid sequence selected from the group consisting of:
(a) SEQ ID NO: 9,
(b) SEQ ID NO:25,
(c) SEQ ID NO:41, and
(d) SEQ ID NO: 57, and
a Kabat light chain complementarity determining region-2 (VL-CDR2) amino acid sequence which is identical to the VL-CDR2 of the VL region comprising the amino acid sequence selected from the group consisting of:
(a) SEQ ID NO: 9,
(b) SEQ ID NO: 25,
(c) SEQ ID NO: 41, and
(d) SEQ ID NO: 57, and
a Kabat light chain complementarity determining region-3 (VL-CDR3) amino acid sequence which is identical to the VL-CDR3 of the VL region comprising the amino acid sequence selected from the group consisting of:
(a) SEQ ID NO: 9;
(b) SEQ ID NO: 25;
(c) SEQ ID NO: 41; and
(d) SEQ ID NO: 57.

4. The antibody of claim 1 which comprises:
a Kabat heavy chain complementarity determining region-1 (VH-CDR1) comprising an amino acid sequence selected from the group consisting of:
(a) SEQ ID NO: 12,
(b) SEQ ID NO: 28,
(c) SEQ ID NO: 44, and
(d) SEQ ID NO: 60,
a Kabat heavy chain complementarity determining region-2 (VH-CDR2) comprising an amino acid sequence selected from the group consisting of:
(a) SEQ ID NO: 13,
(b) SEQ ID NO: 29,
(c) SEQ ID NO: 45, and
(d) SEQ ID NO: 61, and
a Kabat heavy chain complementarity determining region-3 (VH-CDR3) comprising an amino acid sequence selected from the group consisting of
(a) SEQ ID NO: 14,
(b) SEQ ID NO: 30,
(c) SEQ ID NO: 46, and
(d) SEQ ID NO: 62, and
a Kabat light chain complementarity determining region-1 (VL-CDR1) comprising an amino acid sequence selected from the group consisting of:
(a) SEQ ID NO: 15,
(b) SEQ ID NO: 31,
(c) SEQ ID NO: 47, and
(d) SEQ ID NO: 63, and
a Kabat light chain complementarity determining region-2 (VL-CDR2) comprising an amino acid sequence selected from the group consisting of:
(a) SEQ ID NO: 16,
(b) SEQ ID NO: 32,
(c) SEQ ID NO: 48, and
(d) SEQ ID NO: 64, and
a Kabat light chain complementarity determining region-3 (VL-CDR3) comprising an amino acid sequence selected from the group consisting of:
(a) SEQ ID NO: 17,
(b) SEQ ID NO: 33,
(c) SEQ ID NO: 49, and
(d) SEQ ID NO: 65.

5. The antibody of claim 1 which comprises:
a VH chain comprising an amino acid sequence selected from the group consisting of:
(a) SEQ ID NO: 8,
(b) SEQ ID NO: 24,
(c) SEQ ID NO: 40, and
(d) SEQ ID NO: 56, and
a VL chain comprising an amino acid sequence selected from the group consisting of:
(a) SEQ ID NO: 9,
(b) SEQ ID NO: 25,
(c) SEQ ID NO: 41, and
(d) SEQ ID NO: 57.

6. An antibody which binds the same epitope(s) as the antibody (a), (b), (c) and (d) as defined in claim 3.

7. The antibody of claim 1, which comprises a light chain constant region selected from the group consisting of a human kappa constant region and a human lambda constant region.

8. The antibody of claim 1, which comprises a heavy chain constant region or fragment thereof, wherein the heavy chain constant region is a human IgG1 heavy chain constant region, a human IgG2 heavy chain constant region, a human IgG3 heavy chain constant region, a human IgG4 heavy chain constant region, a human IgM heavy chain constant region, a human IgA1 heavy chain constant region, a human IgA2 heavy chain constant region, a human IgE heavy chain constant region, or a human IgD heavy chain constant region.

9. The antibody of claim 1, wherein said antibody is a chimeric antibody, a human or humanized antibody, and/or a monoclonal antibody.

10. The antibody of claim 1, wherein said antibody is an antagonist of IL-17B.

11. The antibody of claim 1, which is an antibody fragment directed against IL-17B, wherein the antibody fragment is selected from the group consisting of Fv, Fab, F(ab′)2, Fab′, dsFv, scFv, sc(Fv)2, and diabodies.

12. An isolated nucleic acid comprising a sequence encoding the antibody of claim 1.

13. A composition comprising an isolated nucleic acid sequence encoding a VL region and an isolated nucleic acid encoding a VH region of the antibody of claim 1.

14. A vector comprising an isolated nucleic acid claim 12.

15. A host cell comprising an isolated nucleic acid sequence of claim 12.

16. A pharmaceutical composition comprising the antibody of claim 1 and a pharmaceutically acceptable carrier.

17. An in vitro method of diagnosis comprising measuring a level of an IL-17B using the antibody of claim 1.

18. A method of treating an autoimmune disease, a chronic inflammatory disease, or cancer in a subject in need, comprising administering to the subject a therapeutically effective amount of the antibody of claim 1.

19. A method of treating cancer in a subject in need, comprising administering to the subject a therapeutically effective amount of the antibody of claim 1, wherein said method increases the effectiveness of a therapeutic agent and/or prevents or treats tumor metastasis.

20. A method of producing an antibody that specifically binds IL-17B, comprising culturing the host cell of claim 15 under conditions suitable for expressing said antibody, and recovering said antibody.
